(19) United States
(12) Patent Application Publication
(10) Pub. No.: US 2020/0030202 A1
(43) Pub. Date: Jan. 30, 2020

(54) TWO-STEP TREATMENT FOR DESQUAMATION OF SKIN

(71) Applicant: L'OREAL, Paris (FR)

(72) Inventors: Rebecca Chen, Princeton, NJ (US); Anne-Laure Suzanne Bernard, New-York, NY (US); Seung Yang, Clark, NJ (US)

(21) Appl. No.: 16/049,310

(22) Filed: Jul. 30, 2018

(51) Int. Cl. A61K 8/25; A61K 8/19; A61K 8/44; A61K 8/43; A61K 8/41; A61K 8/365; A61K 8/368; A61Q 19/00

(52) U.S. Cl. CPC A61K 8/25; A61K 8/19; A61K 8/44; A61K 8/43; A61K 2800/28; A61K 8/365; A61K 8/368; A61Q 19/00; A61K 8/41

(57) ABSTRACT

The instant disclosure relates to methods for treating skin, for example, methods for desquamation of the skin of the face. The methods use a two-step process of first treating the skin with an alkaline composition having a pH of at least 8 followed by a second step of treating the skin with an acidic composition having a pH of 5 of less. The acidic composition includes one or more α- and/or β-hydroxy acids, which react with the skin to cause desquamation and stimulate new skin cell growth. Treating the skin with the claimed alkaline compositions improves the efficacy of the treatment with the acidic composition while reducing skin irritation and discomfort.

### FIELD OF THE DISCLOSURE

The instant disclosure relates to methods for treating skin, for example, methods for desquamation of the skin of the face. The methods use a two-step process of first treating the skin with an alkaline composition followed by a second step of treating the skin with an acidic composition that includes one or more α- and/or β-hydroxy acids.

### BACKGROUND

Skin renewal can be stimulated by removal of the outer keratinous layer. Such removal can be effected mechanically, for example, by rubbing with an abrasive composition, or chemically. Chemical peeling uses active chemical agents that interact with the complex structure of the skin, removing the outer layer (stratum corneum for superficial peels, epidermis and possibly dermis for medium and deep peels) to induce a controlled injury to the skin and stimulate skin regeneration. The regenerated skin is smoother and appears younger. Peeling is commonly used to treat visible and/or tactile irregularities of the human skin, to attenuate defects of pigmentation such as skin discoloration and the marks due to acne or varicella, and to smooth irregularities in the texture, in particular fine lines and wrinkles.

Various acids have been used as the active chemical agents for chemical peels. Common acids include α-hydroxy acids (AHA, such as glycolic acid), β-hydroxy acid (BHA, salicylic acid for example), tricholoracetic acid (TCA) and retinoic acid. TCA and retinoic acid are usually used in medium to deep peels and performed in dermatologist office or in a medical spa setting. AHA and BHA can be used as home peels or superficial peels and medium or deep peels depending on the concentration. These acids deliver skin benefits via interacting with the skin to accelerate desquamation and stimulate new skin cell growth.

Higher concentrations of acids improve the efficacy of a peel but often cause discomfort and irritation (e.g., reddening, tingling, burning sensation), and require longer healing times. Even superficial peels cause burning sensations and excessive skin peeling that can last for days. Medium and deep peels are more intense. Recovery from a medium or deep peel generally requires staying indoors for multiple days, and patients must wait several months before receiving a subsequent treatment.

### SUMMARY OF THE DISCLOSURE

The instant case relates to methods for treating skin with a and/or 8-hydroxy acids. The methods provide improved efficacy with less irritation and discomfort. This is accomplished using a unique two-step process, wherein the skin is treated with an alkaline treatment prior to being treated with the acidic treatment (the acidic chemical peel). The inventors surprisingly found that pre-treating the skin with an alkaline composition improves the efficacy of the acidic chemical peel and reduces irritation and discomfort. Reversing the order (i.e., treating the skin with the acidic chemical peel first followed by treatment with an alkaline composition) did not produce similar results. Instead, the results are no better and sometimes even worse than treating the skin with only the acid chemical peel.

More specifically, the methods of the instant disclosure include applying an alkaline composition having a pH of at least 8 to the skin and allowing it to remain on the skin for a first period of time, for example, a period of time of at least 30 seconds, the alkaline composition comprising:
(i) about 0.05 to about 25 wt. %, based on the total weight of the alkaline composition, of one or more alkaline agents; and
(ii) optionally, about 0.1 to about 10 wt. %, based on the total weight of the alkaline composition, of one or more thickeners;
(iii) optionally, about 0.05 to about 1 wt. %, based on the total weight of the alkaline composition, of one or more colorants;
(ii) optionally, about 0.5 to about 25 wt. %, based on the total weight of the alkaline composition, of one or more water-soluble solvents; and
(iii) water.

After allowing the alkaline composition to remain on the skin for the first period of time, an acidic composition having a pH of 5 or less is applied to the skin and allowed to remain on the skin for a second period, for example, a second period of time of at least 30 seconds, the acidic composition comprising:
(i) about 1 to about 25 wt. %, based on the total weight of the acidic composition, of one or more α- and/or β-hydroxy acids;
(ii) optionally, about 0.1 to about 25 wt. %, based on the total weight of the acidic composition, of one or more whitening actives, such as arbutin, azelaic acid, linoleic acid, kojic acid, N-acetyl-4,S-cysteaminylphenol, niacinamide, resorcinol;
(iii) optionally, about 0.1 to about 25 wt. %, based on the total weight of the acidic composition, of one or more antioxidants, such as ascorbic acid, Vitamin E;
(iv) optionally, about 0.1 to about 10 wt. %, based on the total weight of the acidic composition, of one or more thickeners;
(v) optionally, about 0.05 to about 1 wt. %, based on the total weight of the acidic composition, of one or more colorants;
(ii) optionally, about 1 to about 25 wt. %, based on the total weight of the acidic composition, of one or more water-soluble solvents; and
(iii) water.

The methods of the disclosure are useful for desquamation of the skin; and are useful for improving the radiance of the skin, improving the evenness of skin tone, improving the clarity of skin, reducing the appearance of fine lines and/or wrinkles, and improving the overall appearance of the skin.

### BRIEF DESCRIPTION OF THE DRAWINGS

Implementations of the present technology will now be described, by way of example only, with reference to the attached figures, wherein:

FIG. 1 graphically shows the shifts of the melting transition temperatures measured by Differential Scanning Calorimetry (DSC) associated with treatment-induced disordering of intercellular lipids in stratum corneum;

FIG. 2 graphically shows and compares the lipid disorganization results obtained using a positive control (1), an alkaline treatment only (2), an acidic treatment only (3), an acidic treatment followed by an alkaline treatment (4), and an alkaline treatment followed by an acidic treatment (5);

FIG. 3 graphically shows and compares the lipid disorganization results obtained using a positive control (1), an acidic treatment only (2), alkaline treatments only (3 and 6), various alkaline treatments followed by acidic treatments (4, 7, and 9), and acidic treatments followed by various alkaline treatments (5 and 8);

FIG. 4 graphically shows and compares total protein recovered from areas with an untreated control (1), an acidic treatment only (2), and various alkaline treatments followed by an acidic treatment (3, 4, 5, and 6).

It should be understood that the various aspects are not limited to the arrangements and instrumentality shown in the drawings.

### DETAILED DESCRIPTION OF THE DISCLOSURE

The methods of the instant disclosure include application of an alkaline composition having a pH of at least 8 to the skin and allowing it to remain on the skin for a first period of time, for example, a period of time of at least 30 seconds, the alkaline composition comprising:
(i) about 0.05 to about 25 wt. %, based on the total weight of the alkaline composition, of one or more alkaline agents; and
(ii) optionally, about 0.1 to about 10 wt. %, based on the total weight of the alkaline composition, of one or more thickeners;
(iii) optionally, about 0.05 to about 1 wt. %, based on the total weight of the alkaline composition, of one or more colorants;
(ii) optionally, about 0.5 to about 25 wt. %, based on the total weight of the alkaline composition, of one or more water-soluble solvents; and
(iii) water.

After allowing the alkaline composition to remain on the skin for the first period of time, an acidic composition having a pH of 5 or less is applied to the skin and allowed to remain on the skin for a second period, for example, a second period of time of at least 30 seconds, the acidic composition comprising:
(i) about 1 to about 25 wt. %, based on the total weight of the acidic composition, of one or more α- and/or β-hydroxy acids;
(ii) optionally, about 0.1 to about 25 wt. %, based on the total weight of the acidic composition, of one or more whitening actives, such as arbutin, azelaic acid, linoleic acid, kojic acid, N-acetyl-4,S-cysteaminylphenol, niacinamide, resorcinol;
(iii) optionally, about 0.1 to about 25 wt. %, based on the total weight of the acidic composition, of one or more antioxidants, such as ascorbic acid, Vitamin E;
(iv) optionally, about 0.1 to about 10 wt. %, based on the total weight of the alkaline composition, of one or more thickeners;
optionally, about 0.05 to about 1 wt. %, based on the total weight of the alkaline composition, of one or more colorants; (ii) optionally, about 1 to about 25 wt. %, based on the total weight of the acidic composition, of one or more water-soluble solvents; and
(iii) water.

The first step of applying an alkaline composition is to the skin may be referred to as an “alkaline treatment,” and the second step of applying an acidic composition to the skin may be referred to as an “acidic treatment.”

The length of time that the alkaline composition and the acidic composition are allowed to remain on the skin can vary. Nonetheless, as noted above, these compositions are usually allowed to remain on the skin for at least 30 seconds, and may be allowed to remain on the skin for long periods of time, for example they may be allowed to remain on the skin for up to 1 day (about 24 hours) without rinsing or washing. In some cases, it may be preferable to rinse or wash the alkaline composition from the skin before application of the acidic composition but in some cases there is no need to rinse or wash the alkaline composition from the skin. In some instances, it is preferable to apply the acidic composition onto the skin without first rinsing or washing the alkaline composition from the skin. Furthermore, the amount of time that the alkaline composition is allowed to remain on the skin and the acidic composition is allowed to remain on the skin is independent of each other and may vary greatly.

Typically, the alkaline composition is applied to the skin and allowed to remain on the skin for about 30 seconds to about 1 day (about 24 hours) before application of the acidic composition. The alkaline compositions can optionally be washed or rinsed from the skin prior to application of the acidic composition or the acidic composition can be directly applied to the skin onto which the alkaline composition has been applied. In some instances, the alkaline composition can be applied to the skin and allowed to remain on the skin for about 1 minute to about 1 day (24 hours), about 1 minute to about a half of a day (about 12 hours), from about 1 minute to about 2 hours, from about 1 minute to about 1 hour, or about 1 minute to about 30 minutes before application of the acidic composition. Again, the alkaline composition can optionally be washed or rinsed from the skin prior to application of the acidic composition or the acidic composition can be directly applied to the skin onto which the alkaline composition has been applied without first washing or rinsing the alkaline composition from the skin.

In instances when the alkaline composition is rinsed or washed from the skin prior to application of the acidic composition, the acidic composition may be immediately applied to the skin after rinsing or washing. Alternatively, the alkaline composition can be rinsed or washed from the skin without immediately applying the acidic composition. For example, the acidic composition may be applied to the skin within about 1 day (about 24 hours) after rinsing or washing the alkaline composition from the skin. Similarly, the acidic composition may be applied to the skin within about a half day (about 12 hours), about 6 hours, about 4 hours, about 2 hours, about 1 hour, about 30 minutes, about 15 minutes, about 10 minutes or about 5 minutes after rinsing or washing the alkaline composition from the skin.

Typically, the acidic composition is applied to the skin and allowed to remain on the skin for about 30 seconds to about 1 day (about 24 hours) before rinsing or washing the skin. In some instances, the acidic composition can be applied to the skin and allowed to remain on the skin for about 1 minute to about 1 day (24 hours), about 1 minute to about a half of a day (about 12 hours), from about 1 minute to about 2 hours, from about 1 minute to about 1 hour, or about 1 minute to about 30 minutes before rinsing or washing the skin.

The methods for treating skin are particularly useful for treating the skin of the face and neck, in particular, the face and neck of human individuals. The methods provide for desquamation of the skin; and are useful for improving the radiance of the skin, improving the evenness of skin tone, improving the clarity of skin, reducing the appearance of fine lines and/or wrinkles, and improving the overall appearance of the skin.

Alkaline Compositions

The alkaline compositions of the instant disclosure include one or more alkaline agents; optionally, one or more water-soluble solvents; and water. An alkaline agent is a compound that alkalizes the compositions (increases pH to a desired level). The amount of alkaline agents in the alkaline composition can vary depending on the desired pH. A higher pH usually requires stronger or more alkaline agents. Nonetheless, the total amount of alkaline agents in the alkaline composition is typically about 0.05 to about 25 wt. %, based on the total weight of the alkaline composition. In some cases, the total amount of alkaline agents in the alkaline composition is about 0.05 to about 20 wt. %, about 0.05 to about 15 wt. %, about 1 to about 10 wt. %, about 2 to about 25 wt. %, about 2 to about 20 wt. %, about 2 to about 15 wt. %, about 2 to about 10 wt. %, about 3 to about 20 wt. %, about 3 to about 15 wt. %, or about 3 to about 10 wt. %, based on the total weight of the alkaline composition.

Many alkaline agents are known and can be used in the alkaline composition. Non-limiting examples of alkaline agents include alkali metal hydroxides (e.g., lithium hydroxide, sodium hydroxide, potassium hydroxide, magnesium hydroxide, magnesium oxide), alkaline-earth metal hydroxides (e.g., calcium hydroxide), alkali metal silicates, (e.g., sodium silicate, lithium silicate, and potassium silicate, magnesium silicate, aluminum silicate, fluorphlogopite), alkali metal carbonates (e.g., lithium carbonate, sodium carbonate, potassium carbonate, sodium bicarbonate), alkaline-earth metal carbonates (e.g., calcium carbonate), organic carbonates (e.g., guanidine carbonate), basic amino acids (arginine, lysine, histidine), and their polymers (poly arginine, poly lysine, etc.), organic amines, such as alkanolamines (e.g., monoethanolamine, diethanolamine, triethanolamine, aminomethyl propanol), ammonium hydroxide, and a mixture thereof. A more exhaustive but non-limiting description of alkaline agents that may be included in the alkaline compositions is provided later, under the heading “Alkaline Agents.”

The pH of the alkaline composition is typically at least 8, but not usually higher than 13. For example, in some instances, the pH of the alkaline composition is at least 8 to about 13, at least 9 to about 13, at least 10 to about 13, about 11 to about 13, or about 12 to about 13. In some instances it is preferable that the pH of the alkaline compositions is about 10 to about 12.5, or about 10.5 to about 12.5, or about 11 to about 12.5, about 11.5 to about 12.5, or about 12.

The alkaline compositions may optionally include one or more water soluble solvents. Non-limiting examples of water soluble solvents include monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds. A more exhaustive but non-limiting description of water soluble solvents that may be included in the alkaline compositions is provided later, under the heading “Water Soluble Solvents.”

The total amount of water soluble solvent in the alkaline compositions can vary depending on the type and form of the compositions. For example, the alkaline compositions may include less water-soluble solvents when they are in the form of an emulsion than when they are in other form, such as solutions or gels.

The total amount of water-soluble solvents in the alkaline compositions may be from about 0.5 to about 25 wt. %, based on the total weight of the alkaline composition. In some cases, the total amount of the water soluble solvents is from about 0.5 to about 20 wt. %, about 0.5 to about 15 wt. %, about 1 to about 25 wt. %, about 1 to about 20 wt. %, or about 1 to about 15 wt. %. If the alkaline composition is in the form of an emulsion, it may not include water soluble solvents. Nonetheless, emulsions may optionally include water soluble solvents in the amounts set forth above, but can also be in lower amounts. For example, an alkaline composition in the form of an emulsion can have from about 0.5 to about 15 wt. %, about 0.5 to about 10 wt. %, about 0.5 to about 5 wt. %, about 1 to about 15 wt. %, about 1 to about 10 wt. %, or about 1 to about 5 wt. % of water soluble solvents based on the total weight of the alkaline composition in the form of an emulsion. For non-emulsified alkaline compositions, the total amount of water soluble solvents can be the amounts set forth above but can also be in higher amounts. For example, the total amount of water soluble solvents in non-emulsified alkaline compositions can be from about 2 to about 25 wt. % about 2 to about 20 wt. %, about 2 to about 15 wt. %, about 3 to about 25 wt. %, about 3 to about 20 wt. %, about 3 to about 15 wt. %, about 5 to about 25 wt. %, about 5 to about 20 wt. %, or about 5 to about 15 wt. %, based on the total weight of the non-emulsified alkaline composition.

The alkaline composition described throughout this disclosure may be in any suitable physical form. Suitable forms include, but are not limited to emulsified or non-emulsified liquids, lotions, milks, mousses, sprays, gels, creams, pastes, and the like. Regardless of the form of the alkaline composition, the alkaline composition typically includes water, and typically includes a significant amount of water, e.g., at least 20 wt. %, based on the total weight of the alkaline composition.

In some cases, the alkaline composition is a non-emulsified liquid. Non-emulsified liquid alkaline compositions can be very simple formulations that do not require many additional components such as emulsifiers, non-silicone fatty compounds, silicones, surfactants, thickening agents, etc. These additional components can certainly be included in non-emulsified liquid alkaline compositions but they are not required. Therefore, these components can be excluded from the non-emulsified liquid alkaline compositions, e.g., the non-emulsified liquid alkaline compositions can be free or essentially free of emulsifiers and/or free or essentially free of non-silicone fatty compounds and/or free or essentially free of silicones and/or free or essentially free of surfactants and/or free or essentially free of thickening agents, etc.

Generally, the total amount of water in non-emulsified liquid alkaline compositions is higher than the total amount of water in emulsified alkaline compositions. For example, the total amount of water in non-emulsified liquid alkaline compositions can be at least 20 to about 95 wt. %, as discussed above, but can also be about 50 to about 95 wt. %, about 60 to about 95 wt. %, about 65 to about 95 wt. %, about 70 to about 95 wt. %, about 75 to about 95 wt. %, about 50 to about 92 wt. %, about 60 to about 92 wt. %, about 65 to about 92 wt. %, about 70 to about 92 wt. %, about 75 to about 92 wt. %, based on the total weight of the non-emulsified liquid alkaline composition.

In one embodiment, non-emulsified liquid alkaline compositions include:
(i) about 0.05 to about 25 wt. %, preferably about 2 to about 20 wt. %, more preferably about 3 to about 15 wt. %, based on the total weight of the alkaline composition, of one or more alkaline agents such as alkali metal hydroxides, alkaline-earth metal hydroxides, alkali metal silicates, alkali metal carbonates, alkaline-earth metal carbonates, organic carbonates, basic amino acids, and their polymers, organic amines, such as alkanolamines, ammonium hydroxide, and a mixture thereof; and
(ii) about 0.5 to about 25 wt. %, preferably about 1 to about 20 wt. %, more preferably about 5 to about 15 wt. %, based on the total weight of the alkaline composition, of one or more water-soluble solvents such as glycerin, mono-alcohols, polyols (polyhydric alcohols), glycols, and a mixture thereof, preferably glycerin; and
(iii) about 50 to about 95 wt. %, preferably about 65 to about 95 wt. %, more preferably about 75 to about 92 wt. %, based on the total weight of the alkaline composition, of water.

The non-emulsified liquid alkaline compositions of the above-embodiment can optionally be free or essentially free of emulsifiers and/or free or essentially free of non-silicone fatty compounds and/or free or essentially free of silicones and/or free or essentially free of surfactants and/or free or essentially free of thickening agents, etc.

In some cases, the alkaline composition is a non-emulsified gel. A thickening agent or gelling agent is often used to form a gel. Therefore, the non-emulsified alkaline compositions in the form of a gel may include one or more thickening agents. Non-limiting examples of thickening agents include carboxylic acid polymers, crosslinked polyacrylate polymers, polyacrylamide polymers, polysaccharides, gums, and a mixture thereof. A more exhaustive but non-limiting description of thickening agents that may be included in the alkaline compositions is provided later, under the heading “Thickening Agents.”

The total amount of thickening agents in non-emulsified alkaline compositions in the form of a gel can vary but is typically about 0.1 to about 10 wt. %, based on the total weight of the non-emulsified alkaline composition in the form of a gel. In some cases, the total amount of thickening agent may be about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, or about 1 to about 5 wt. %, based on the total weight of the non-emulsified alkaline composition in the form of a gel.

The non-emulsified alkaline compositions in the form of a gel can optionally include one or more non-silicone fatty compounds. Non-limiting examples of non-silicone fatty compounds include oils, mineral oil, alkanes (paraffins), fatty alcohols, fatty acids, fatty alcohol derivatives, fatty acid derivatives, esters of fatty alcohols, hydroxy-substituted fatty acids, waxes, triglyceride compounds, lanolin, and a mixture thereof. A more exhaustive but non-limiting description of non-silicone fatty compounds that may be included is provided later, under the heading “Non-Silicone Fatty Compounds.”

The total amount of non-silicone fatty compound in the non-emulsified alkaline compositions in the form of a gel, if present, can vary but are typically in an amount of about 0.01 to about 15 wt. %, based on the total weight of the non-emulsified alkaline composition in the form of a gel. In some cases, the total amount of non-silicone fatty compounds is about 0.01 to about 12 wt. %, about 0.01 to about 10 wt. %, about 0.01 to about 8 wt. %, about 0.01 to about 5 wt. %, about 0.1 to about 15 wt. %, about 0.1 to about 12 wt. %, about 0.1 to about 10 wt. %, about 0.1 to about 8 wt. %, about 0.1 to about 5 wt. %, about 1 to about 15 wt. %, about 1 to about 12 wt. %, about 1 to about 10 wt. %, about 1 to about 8 wt. %, or about 1 to about 5 wt. %, based on the total weight of the non-emulsified alkaline composition in the form of a gel.

In one embodiment non-emulsified alkaline composition in the form of a gel include:
(i) about 0.05 to about 25 wt. %, preferably about 2 to about 20 wt. %, more preferably about 3 to about 15 wt. %, based on the total weight of the alkaline composition, of one or more alkaline agents such as alkali metal hydroxides, alkaline-earth metal hydroxides, alkali metal silicates, alkali metal carbonates, alkaline-earth metal carbonates, organic carbonates, basic amino acids, and their polymers, organic amines, such as alkanolamines, ammonium hydroxide, and a mixture thereof; and
(ii) optionally, about 0.5 to about 25 wt. %, preferably about 1 to about 20 wt. %, more preferably about 5 to about 15 wt. %, based on the total weight of the alkaline composition, of one or more water-soluble solvents such as glycerin, mono-alcohols, polyols (polyhydric alcohols), glycols, and a mixture thereof, preferably glycerin;
(iii) about 50 to about 95 wt. %, preferably about 65 to about 95 wt. %, more preferably about 75 to about 92 wt. %, based on the total weight of the alkaline composition, of water;
(iv) about 0.1 to about 10 wt. %, preferably about 0.5 to about 8, more preferably about 1 to about 5 wt. %, based on the total weight of the alkaline composition, of one or more thickening agents, for example, carboxylic acid polymers, crosslinked polyacrylate polymers, polyacrylamide polymers, polysaccharides, gums, and a mixture thereof (and in some cases, preferably acrylamide/sodium acryloyldimethyltaurate copolymer); and
(v) about 0.01 to about 15 wt. %, preferably about 1 to about 12 wt. %, more preferably about 1 to about 10 wt. %, based on the total weight of the alkaline composition, of one or more non-silicone fatty compounds, for example, oils, mineral oil, alkanes (paraffins), fatty alcohols, fatty acids, fatty alcohol derivatives, fatty acid derivatives, esters of fatty alcohols, hydroxy-substituted fatty acids, waxes, triglyceride compounds, lanolin, and a mixture thereof (and in some cases, preferably one or more alkanes (paraffins) such as isohexadecane).

The non-emulsified alkaline compositions in the form of a gel of the above-embodiment can optionally be free or essentially free of silicones.

In some instances, the alkaline compositions are in the form of an emulsion. The emulsions include an aqueous phase and a fatty phase. The fatty phase includes one or more non-silicone fatty compounds. Non-limiting examples of non-silicone fatty compounds include oils, mineral oil, alkanes (paraffins), fatty alcohols, fatty acids, fatty alcohol derivatives, fatty acid derivatives, esters of fatty alcohols, hydroxy-substituted fatty acids, waxes, triglyceride compounds, lanolin, and a mixture thereof. A more exhaustive but non-limiting description of non-silicone fatty compounds that may be included is provided later, under the heading “Non-Silicone Fatty Compounds.”

The total amount of non-silicone fatty compounds in the emulsified alkaline compositions can vary but is typically in an amount of about 10 to about 85 wt. %, based on the total weight of the emulsified alkaline composition. In some cases, the total amount of non-silicone fatty compounds in the emulsified alkaline composition is about 10 to about 85 wt. %, about 20 to about 85 wt. %, about 30 to about 85 wt. %, about 40 to about 80 wt. %, about 50 to about 80 wt. %, about 60 to about 80 wt. %, about 10 to about 75 wt. %, about 20 to about 75 wt. %, about 30 to about 75 wt. %, about 40 to about 75 wt. %, about 50 to about 75 wt. %, about 60 to about 75 wt. %, about 10 to about 70 wt. %, about 20 to about 70 wt. %, about 30 to about 70 wt. %, about 40 to about 70 wt. %, about 50 to about 70 wt. %, about 60 to about 70 wt. %, about 10 to about 60 wt. %, about 20 to about 60 wt. %, about 30 to about 60 wt. %, or about 40 to about 60 wt. %, based on the total amount of the emulsified alkaline composition.

The amount of water in emulsified alkaline composition is generally lower than the amount of water in non-emulsified alkaline compositions (due to the inclusion of a substantial fatty phase in an emulsified alkaline composition). The total amount of water in the emulsified alkaline compositions can vary but may be from about 15 to about 70 wt. %, based on the total weight of the emulsified alkaline composition. In some cases, the total amount of water in the emulsified alkaline composition is about 20 to about 70 wt. %, about 25 to about 70 wt. %, about 15 to about 60 wt. %, about 20 to about 60 wt. %, about 25 to about 60 wt. %, about 15 to about 50 wt. %, about 20 to about 50 wt. %, about 25 to about 50 wt. %, about 15 to about 40 wt. %, about 20 to about 40 wt. %, or about 25 to about 40 wt. %, based on the total weight of the emulsified alkaline composition.

The emulsified alkaline compositions include one or more emulsifiers. Many emulsifiers are known in the art and may be used, including, amphoteric, anionic, cationic, and nonionic emulsifiers. In some instances, it is useful to include at least one nonionic emulsifier. Non-limiting examples of nonionic emulsifiers include polyol esters, a glycerol ethers, oxyethylenated and/or oxypropylenated ethers, ethylene glycol polymers, glucoside surfactants (such as decyl glucoside), polysorbates, sorbitan oleate, and mixtures thereof. In some instances, an oxyalkylenated organosiloxane emulsifier is included. Non-limiting examples include dimethicone/PEG-10/15 crosspolymer, PEG-15 lauryl dimethicone crosspolymer, PEG-15 lauryl dimethicone crosspolymer, PEG-10 lauryl dimethicone crosspolymer, or a mixture thereof. A more exhaustive but non-limiting list of emulsifiers that may be included in the cosmetic compositions is provided later, under the heading “Emulsifiers.”

The total amount of emulsifiers in the emulsified alkaline composition can vary but is typically about 0.1 to about 15 wt. %, based on the total weight of the emulsified alkaline composition. In some cases, the total amount of emulsifiers is about 0.1 to about 10 wt. %, about 0.1 to about 8 wt. %, about 0.1 to about 5 wt. %, about 0.5 to about 15 wt. %, about 0.5 to about 10 wt. %, about 0.5 to about 8 wt. %, about 0.5 to about 5 wt. %, about 1 to about 15 wt. %, about 1 to about 10 wt. %, about 1 to about 8 wt. %, or about 1 to about 5 wt. %, based on the total amount of the emulsified alkaline composition.

A thickening agent can optionally be included in emulsified alkaline compositions. Non-limiting examples of thickening agents include carboxylic acid polymers, crosslinked polyacrylate polymers, polyacrylamide polymers, polysaccharides, gums, celluloses including cetyl hydroxyethylcellulose, and a mixture thereof. A more exhaustive but non-limiting description of thickening agents that may be included in the alkaline compositions is provided later, under the heading “Thickening Agents.”

The total amount of thickening agents in emulsified alkaline compositions can vary but is typically about 0.01 to about 10 wt. %, based on the total weight of the emulsified alkaline composition. In some cases, the total amount of thickening agent may be about 0.01 to about 8 wt. %, about 0.01 to about 5 wt. %, about 0.1 to about 10 wt. %, about 0.1 to about 8 wt. %, or about 0.1 to about 5 wt. %, based on the total weight of the emulsified alkaline composition.

In one embodiment the alkaline compositions in the form of an emulsion include:
(i) about 0.05 to about 25 wt. %, preferably about 2 to about 20 wt. %, more preferably about 3 to about 15 wt. %, based on the total weight of the alkaline composition, of one or more alkaline agents such as alkali metal hydroxides, alkaline-earth metal hydroxides (e.g., calcium hydroxide), alkali metal silicates, alkali metal carbonates, alkaline-earth metal carbonates, organic carbonates, basic amino acids, and their polymers, organic amines, such as alkanolamines, ammonium hydroxide, and a mixture thereof; and
(ii) optionally, about 0.5 to about 25 wt. %, preferably about 1 to about 20 wt. %, more preferably about 5 to about 15 wt. %, based on the total weight of the alkaline composition, of one or more water-soluble solvents such as glycerin, mono-alcohols, polyols (polyhydric alcohols), glycols, and a mixture thereof;
(iii) about 15 to about 75 wt. %, preferably about 20 to about 70 wt. %, more preferably about 20 to about 60 wt. %, based on the total weight of the alkaline composition, of water;
(iv) about 10 to about 85 wt. %, preferably about 45 to about 80 wt. %, more preferably about 50 to about 75 wt. % of one or more non-silicone fatty compounds, for example, oils, mineral oil, alkanes (paraffins), fatty alcohols, fatty acids, fatty alcohol derivatives, fatty acid derivatives, esters of fatty alcohols, hydroxy-substituted fatty acids, waxes, triglyceride compounds, lanolin, and a mixture thereof (and in some cases, preferably one or more oils such as mineral oil;
(v) about 0.01 to about 10 wt. %, preferably about 0.1 to about 8 wt. %, more preferably about 1 to about 5 wt. %, based on the total weight of the alkaline composition, of one or more emulsifiers, preferably one or more nonionic emulsifiers; and
(vi) optionally, about 0.1 to about 10 wt. %, preferably about 0.5 to about 8, more preferably about 1 to about 5 wt. %, based on the total weight of the alkaline composition, of one or more thickening agents, for example, carboxylic acid polymers, crosslinked polyacrylate polymers, polyacrylamide polymers, polysaccharides, gums, celluloses, and a mixture thereof (and in some cases, preferably celluloses such as cetyl hydroxyethylcellulose).

Acidic Compositions

The acidic compositions include: one or more α- and/or β-hydroxy acids; optionally, one or more water-soluble solvents; and water. Alpha hydroxy acids or α-hydroxy acids (AHAs) are a class of chemical compounds that have of a carboxylic acid substituted with a hydroxyl group on the adjacent carbon. A beta hydroxy acid or β-hydroxy acid (BHA) is a class of chemical compounds that having a carboxylic acid functional group and hydroxy functional group separated by two carbon atoms. They are closely related to alpha hydroxy acids, in which the two functional groups are separated by one carbon atom.

The total amount of α- and/or β-hydroxy acids in the acidic composition can vary but is typically about 1 to about 25 wt. %, based on the total weight of the acidic composition. In some cases, the total amount of α- and/or β-hydroxy acids is about 1 to about 20 wt. %, about 1 to about 15 wt. %, about 1 to about 12 wt. %, about 2 to about 25 wt. %, about 2 to about 20 wt. %, about 2 to about 15 wt. %, about 2 to about 12 wt. %, about 5 to about 25 wt. %, about 5 to about 20 wt. %, about 5 to about 15 wt. %, about 5 to about 12 wt. %, or about 8 to about 12 wt. %, based on the total weight of the acidic composition.

Non-limiting examples of α-hydroxy acids include lactic acid, citric acid, glycolic acid, mandelic acid, phytic acid, mandelic acid, benzylic acid, malic acid, tartaric acid, gluconolactone, galactonolactone, glucuronolactone, galacturonolactone, gulonolactone, ribonolactone, saccharic acid lactone, pantoyllactone, glucoheptonolactone, mannonolactone, or galactoheptonolactone

Non-limiting examples of 13 hydroxy acids include salicylic acid, acetylsalicylic acid, β-Hydroxypropionic acid, β-Hydroxybutyric acid, β-Hydroxy β-methylbutyric acid, carnitine, lipohydroxy acid, 3-hydroxy valeric acid, and a mixture thereof.

Alpha keto acids (e.g., pyruvic acid) may also be used.

In some instances, it may be preferable to include two or more α- and/or β-hydroxy acids. Additionally, in some cases it is useful to include at least one α-hydroxy acid and at least one of β-hydroxy acid in the acidic composition. Although any combination of α-hydroxy acids and β-hydroxy acid can be used, a combination of lactic acid and salicylic acid is particularly useful in some cases.

The pH of the acidic composition is typically 5 or less, but is not usually lower than about 2. For example, in some instances, the pH of the acidic composition is about 2 to 5, about 3 to 5, about 2 to about 4, about 3 to about 4, or about 3.5.

The acidic compositions may optionally include one or more water soluble solvents. Non-limiting examples of water soluble solvents include monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds. A more exhaustive but non-limiting description of water soluble solvents that may be included in the alkaline compositions is provided later, under the heading “Water Soluble Solvents.”

The total amount of water soluble solvent in the acidic compositions can vary depending on the type and form of the compositions. The total amount of water-soluble solvents in the acidic compositions may be from about 0.5 to about 40 wt. %, based on the total weight of the acidic composition. In some cases, the total amount of the water soluble solvents is from about 0.5 to about 30 wt. %, about 0.5 to about 25 wt. %, about 0.5 to about 20 wt. %, about 1 to about 40 wt. %, about 1 to about 30 wt. %, or about 1 to about 25 wt. %, about 1 to about 20 wt. %, about 5 to about 40 wt. %, about 5 to about 30 wt. %, about 5 to about 25 wt. % or about 5 to about 20 wt. %, based on the total weight of the acidic composition.

The acidic composition described throughout this disclosure may be in any suitable physical form. Suitable forms include, but are not limited to emulsified or non-emulsified liquids, lotions, milks, mousses, sprays, gels, creams, pastes, and the like. Regardless of the form of the acidic composition, the acidic composition typically includes water, and typically includes a significant amount of water, e.g., at least 20 wt. %, based on the total weight of the acidic composition.

The total amount of water in the acidic compositions can vary depending on the specific form of the composition. Nonetheless, regardless of the form of the compositions, typically the total amount of water is in the range of at least 20 to about 95 wt. %, based on the total weight of the acidic composition. The total amount of water can be from about 25 to about 95 wt. %, about 30 to about 90 wt. %, or about 35 to about 85 wt. %, based on the total weight of the acidic composition.

In some cases, the acidic composition is a non-emulsified liquid. Non-emulsified liquid acidic compositions can be very simple formulations that do not require many additional components such as emulsifiers, non-silicone fatty compounds, silicones, surfactants, thickening agents, etc. These additional components can certainly be included in non-emulsified liquid acidic compositions but they are not required. Therefore, these components can be excluded from the non-emulsified liquid acidic compositions, e.g., the non-emulsified liquid acidic compositions can be free or essentially free of emulsifiers and/or free or essentially free of non-silicone fatty compounds and/or free or essentially free of silicones and/or free or essentially free of surfactants and/or free or essentially free of thickening agents, etc.

Generally, the total amount of water in non-emulsified liquid acidic compositions is higher than the total amount of water in emulsified acidic compositions. For example, the total amount of water in non-emulsified liquid acidic compositions can be at least 20 to about 95 wt. %, as discussed above, but can also be about 50 to about 95 wt. %, about 60 to about 95 wt. %, about 65 to about 95 wt. %, about 70 to about 95 wt. %, about 50 to about 90 wt. %, about 60 to about 90 wt. %, about 65 to about 90 wt. %, about 70 to about 90 wt. %, about 50 to about 85 wt. %, about 60 to about 85 wt. %, or about 70 to about 95 wt. %, based on the total weight of the non-emulsified liquid acidic composition.

In one embodiment, non-emulsified liquid acidic compositions include:
(i) about 1 to about 25 wt. %, preferably about 2 to about 20 wt. %, more preferably about 5 to about 15 wt. %, based on the total weight of the acidic composition, of one or more α- and/or β-hydroxy acids, for example one or more α- and/or β-hydroxy acids selected from lactic acid, citric acid, glycolic acid, mandelic acid, phytic acid, mandelic acid, benzylic acid, malic acid, tartaric acid, gluconolactone, galactonolactone, glucuronolactone, galacturonolactone, gulonolactone, ribonolactone, saccharic acid lactone, pantoyllactone, glucoheptonolactone, mannonolactone, galactoheptonolactone, 13 hydroxy acids include salicylic acid, acetylsalicylic acid, β-Hydroxypropionic acid, β-Hydroxybutyric acid, β-Hydroxy β-methylbutyric acid, carnitine, lipohydroxy acid, 3-hydroxy valeric acid, and a mixture thereof;
(ii) about 1 to about 25 wt. %, preferably about 2 to about 20 wt. %, more preferably about 5 to about 20 wt. %, based on the total weight of the acidic composition, of one or more water-soluble solvents, such as glycerin, mono-alcohols, polyols (polyhydric alcohols), glycols, and a mixture thereof, preferably a C1-C4 mono-alcohol and/or glycerol; and
(iii) about 50 to about 95 wt. %, preferably about 60 to about 90 wt. %, more preferably about 65 to about 85 wt. % of water.

The non-emulsified liquid acidic compositions of the above-embodiment can optionally be free or essentially free of emulsifiers and/or free or essentially free of non-silicone fatty compounds and/or free or essentially free of silicones and/or free or essentially free of surfactants and/or free or essentially free of thickening agents, etc.

In some cases, the acidic composition is a non-emulsified gel. A thickening agent or gelling agent is often used to form a gel. Therefore, the non-emulsified acidic compositions in the form of a gel may include one or more thickening agents. Non-limiting examples of thickening agents include carboxylic acid polymers, crosslinked polyacrylate polymers, polyacrylamide polymers, polysaccharides, gums, and a mixture thereof. A more exhaustive but non-limiting description of thickening agents that may be included in the acidic compositions is provided later, under the heading “Thickening Agents.”

The total amount of thickening agents in non-emulsified acidic compositions in the form of a gel can vary but is typically about 0.1 to about 10 wt. %, based on the total weight of the non-emulsified acidic composition in the form of a gel. In some cases, the total amount of thickening agent may be about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, or about 1 to about 5 wt. %, based on the total weight of the non-emulsified acidic composition in the form of a gel.

The non-emulsified acidic compositions in the form of a gel can optionally include one or more non-silicone fatty compounds. Non-limiting examples of non-silicone fatty compounds include oils, mineral oil, alkanes (paraffins), fatty alcohols, fatty acids, fatty alcohol derivatives, fatty acid derivatives, esters of fatty alcohols, hydroxy-substituted fatty acids, waxes, triglyceride compounds, lanolin, and a mixture thereof. A more exhaustive but non-limiting description of non-silicone fatty compounds that may be included is provided later, under the heading “Non-Silicone Fatty Compounds.”

The total amount of non-silicone fatty compound in the non-emulsified acidic compositions in the form of a gel, if present, can vary but are typically in an amount of about 0.01 to about 15 wt. %, based on the total weight of the non-emulsified acidic composition in the form of a gel. In some cases, the total amount of non-silicone fatty compounds is about 0.01 to about 12 wt. %, about 0.01 to about 10 wt. %, about 0.01 to about 8 wt. %, about 0.01 to about 5 wt. %, about 0.1 to about 15 wt. %, about 0.1 to about 12 wt. %, about 0.1 to about 10 wt. %, about 0.1 to about 8 wt. %, about 0.1 to about 5 wt. %, about 1 to about 15 wt. %, about 1 to about 12 wt. %, about 1 to about 10 wt. %, about 1 to about 8 wt. %, or about 1 to about 5 wt. %, based on the total weight of the non-emulsified acidic composition in the form of a gel.

In one embodiment non-emulsified acidic composition in the form of a gel include:
(i) about 1 to about 25 wt. %, preferably about 2 to about 20 wt. %, more preferably about 5 to about 15 wt. %, based on the total weight of the acidic composition, of one or more α- and/or β-hydroxy acids, for example one or more α- and/or β-hydroxy acids selected from lactic acid, citric acid, glycolic acid, mandelic acid, phytic acid, mandelic acid, benzylic acid, malic acid, tartaric acid, gluconolactone, galactonolactone, glucuronolactone, galacturonolactone, gulonolactone, ribonolactone, saccharic acid lactone, pantoyllactone, glucoheptonolactone, mannonolactone, galactoheptonolactone, β hydroxy acids include salicylic acid, acetylsalicylic acid, β-Hydroxypropionic acid, β-Hydroxybutyric acid, β-Hydroxy β-methylbutyric acid, carnitine, lipohydroxy acid, 3-hydroxy valeric acid, and a mixture thereof;
(ii) about 1 to about 25 wt. %, preferably about 2 to about 20 wt. %, more preferably about 5 to about 20 wt. %, based on the total weight of the acidic composition, of one or more water-soluble solvents, such as glycerin, mono-alcohols, polyols (polyhydric alcohols), glycols, and a mixture thereof, preferably a C1-C4 mono-alcohol and/or glycerol;
(iii) about 50 to about 95 wt. %, preferably about 60 to about 90 wt. %, more preferably about 65 to about 85 wt. % of water;
(iv) about 0.1 to about 10 wt. %, preferably about 0.5 to about 8, more preferably about 1 to about 5 wt. %, based on the total weight of the acidic composition, of one or more thickening agents, for example, carboxylic acid polymers, crosslinked polyacrylate polymers, polyacrylamide polymers, polysaccharides, gums, and a mixture thereof (and in some cases, preferably a polyacrylate crosspolymer); and
(v) optionally, about 0.01 to about 15 wt. %, preferably about 1 to about 12 wt. %, more preferably about 1 to about 10 wt. % of one or more non-silicone fatty compounds, for example, oils, mineral oil, alkanes (paraffins), fatty alcohols, fatty acids, fatty alcohol derivatives, fatty acid derivatives, esters of fatty alcohols, hydroxy-substituted fatty acids, waxes, triglyceride compounds, lanolin, and a mixture thereof.

The non-emulsified acidic compositions in the form of a gel of the above-embodiment can optionally be free or essentially free of silicones.

In some instances, the acidic compositions are in the form of an emulsion. The emulsions include an aqueous phase and a fatty phase. The fatty phase includes one or more non-silicone fatty compounds. Non-limiting examples of non-silicone fatty compounds include oils, mineral oil, alkanes (paraffins), fatty alcohols, fatty acids, fatty alcohol derivatives, fatty acid derivatives, esters of fatty alcohols, hydroxy-substituted fatty acids, waxes, triglyceride compounds, lanolin, and a mixture thereof. A more exhaustive but non-limiting description of non-silicone fatty compounds that may be included is provided later, under the heading “Non-Silicone Fatty Compounds.”

The total amount of non-silicone fatty compounds in the emulsified acidic compositions can vary but is typically in an amount of about 5 to about 50 wt. %, based on the total weight of the emulsified acidic composition. In some cases, the total amount of non-silicone fatty compounds in the emulsified acidic composition is about 5 to about 40 wt. %, about 5 to about 30 wt. %, about 5 to about 25 wt. %, about 5 to about 20 wt. %, about 10 to about 50 wt. %, about 10 to about 40 wt. %, about 10 to about 30 wt. %, about 10 to about 25 wt. %, or about 10 to about 20 wt. %, based on the total amount of the emulsified acidic composition.

The amount of water in emulsified acidic composition is generally lower than the amount of water in non-emulsified acidic compositions because of the inclusion of the fatty phase in an emulsified acidic composition. The total amount of water in the emulsified acidic compositions can vary but may be from about 15 to about 70 wt. %, based on the total weight of the emulsified acidic composition. In some cases, the total amount of water in the emulsified acidic composition is about 20 to about 70 wt. %, about 25 to about 70 wt. %, about 15 to about 60 wt. %, about 20 to about 60 wt. %, about 25 to about 60 wt. %, about 15 to about 50 wt. %, about 20 to about 50 wt. %, about 25 to about 50 wt. %, about 15 to about 45 wt. %, about 20 to about 45 wt. %, or about 25 to about 45 wt. %, based on the total weight of the emulsified acidic composition.

The emulsified acidic compositions include one or more emulsifiers. Many emulsifiers are known in the art and may be used, including, amphoteric, anionic, cationic, and nonionic emulsifiers. In some instances, it is useful to include at least one organosiloxane emulsifier such as an oxyalkenated organosiloxane emulsifier. Non-limiting examples include dimethicone/PEG-10/15 crosspolymer, PEG-15 lauryl dimethicone crosspolymer, PEG-15 lauryl dimethicone crosspolymer, PEG-10 lauryl dimethicone crosspolymer, PEG-9 polydimethylsiloxyethyl dimethicone, or a mixture thereof. A more exhaustive but non-limiting list of emulsifiers that may be included in the cosmetic compositions is provided later, under the heading “Emulsifiers.”

The total amount of emulsifiers in the emulsified acidic composition can vary but is typically about 0.1 to about 15 wt. %, based on the total weight of the emulsified acidic composition. In some cases, the total amount of emulsifiers is about 0.1 to about 10 wt. %, about 0.1 to about 8 wt. %, about 0.1 to about 5 wt. %, about 0.5 to about 15 wt. %, about 0.5 to about 10 wt. %, about 0.5 to about 8 wt. %, or about 0.5 to about 5 wt. %, based on the total weight of the emulsified acidic composition.

A thickening agent can optionally be used in emulsified acidic compositions. Non-limiting examples of thickening agents include carboxylic acid polymers, crosslinked polyacrylate polymers, polyacrylamide polymers, polysaccharides, gums, celluloses including cetyl hydroxyethylcellulose, and a mixture thereof. A more exhaustive but non-limiting description of thickening agents that may be included in the acidic compositions is provided later, under the heading “Thickening Agents.”

The total amount of thickening agents in an emulsified acidic composition, if present, can vary but is typically about 0.01 to about 10 wt. %, based on the total weight of the emulsified acidic composition. In some cases, the total amount of thickening agent may be about 0.01 to about 8 wt. %, about 0.01 to about 5 wt. %, about 0.1 to about 10 wt. %, about 0.1 to about 8 wt. %, or about 0.1 to about 5 wt. %, based on the total weight of the emulsified acidic composition.

One or more silicones can optionally be included in the emulsified acidic compositions. For example, silicones that may be used include, but are not limited to, polyorganosiloxanes, polyalkylsiloxanes, polyarylsiloxanes, polyalkarylsiloxanes, polyestersiloxanes, and a mixture thereof. Non-limiting examples include dimethicone, cyclomethicone (cyclopentasiloxane), amodimethicone, trimethyl silyl amodimethicone, phenyl trimethicone, trimethyl siloxy silicate, polymethylsilsesquioxane and a mixture thereof.

The total amount of silicones in emulsified acidic compositions, if present, can vary but is typically about 0.1 to about 35 wt. %, based on the total weight of the emulsified acidic composition. In some cases, the total amount of silicones is about 0.1 to about 30 wt. %, about 0.1 to about 20 wt. %, about 1 to about 35 wt. %, about 1 to about 30 wt. %, about 1 to about 20 wt. %, about 5 to about 35 wt. %, about 5 to about 30 wt. %, or about 5 to about 20 wt. %, based on the total weight of the emulsified acidic composition.

In one embodiment the acidic compositions in the form of an emulsion include:
(i) about 1 to about 25 wt. %, preferably about 2 to about 20 wt. %, more preferably about 5 to about 15 wt. %, based on the total weight of the acidic composition, of one or more α- and/or β-hydroxy acids, for example one or more α- and/or β-hydroxy acids selected from lactic acid, citric acid, glycolic acid, mandelic acid, phytic acid, mandelic acid, benzylic acid, malic acid, tartaric acid, gluconolactone, galactonolactone, glucuronolactone, galacturonolactone, gulonolactone, ribonolactone, saccharic acid lactone, pantoyllactone, glucoheptonolactone, mannonolactone, galactoheptonolactone, 13 hydroxy acids include salicylic acid, acetylsalicylic acid, β-Hydroxypropionic acid, β-Hydroxybutyric acid, β-Hydroxy β-methylbutyric acid, carnitine, lipohydroxy acid, 3-hydroxy valeric acid, and a mixture thereof;
(ii) about 1 to about 25 wt. %, preferably about 2 to about 20 wt. %, more preferably about 5 to about 20 wt. %, based on the total weight of the acidic composition, of one or more water-soluble solvents, such as glycerin, mono-alcohols, polyols (polyhydric alcohols), glycols, and a mixture thereof, preferably a C1-C4 mono-alcohol and/or glycerol;
(iii) about 15 to about 70 wt. %, preferably about 20 to about 60 wt. %, more preferably about 25 to about 55 wt. % of water;
(iv) about 5 to about 50 wt. %, preferably about 5 to about 40 wt. %, more preferably about 10 to about 30 wt. %, based on the total weight of the emulsified acidic composition, of one or more non-silicone fatty compounds, for example, oils, mineral oil, alkanes (paraffins), fatty alcohols, fatty acids, fatty alcohol derivatives, fatty acid derivatives, esters of fatty alcohols, hydroxy-substituted fatty acids, waxes, triglyceride compounds, lanolin, and a mixture thereof;
(v) about 0.1 to about 15 wt. %, preferably about 0.1 to about 10 wt. %, more preferably about 0.5 to about 5 wt. %, based on the total weight of the emulsified acidic composition, of one or more emulsifiers, preferably one or more organosiloxane emulsifier; and
(vi) about 0.1 to about 35 wt. %, preferably about 1 to about 30 wt. %, more preferably about 1 to about 20 wt. %, based on the total weight of the emulsified acidic composition, of one or more silicones, for example, dimethicone, cyclomethicone (cyclopentasiloxane), amodimethicone, trimethyl silyl amodimethicone, phenyl trimethicone, trimethyl siloxy silicate, polymethylsilsesquioxane and a mixture thereof.

Alkaline Agents

Many alkaline agents are known and can be used in the alkaline composition. Non-limiting examples of alkaline agents include alkali metal hydroxides (e.g., lithium hydroxide, sodium hydroxide, potassium hydroxide, magnesium hydroxide, magnesium oxide), alkaline-earth metal hydroxides (e.g., calcium hydroxide), alkali metal silicates, (e.g., sodium silicate, lithium silicate, and potassium silicate, magnesium silicate, aluminum silicate, fluorphlogopite), alkali metal carbonates (e.g., lithium carbonate, sodium carbonate, potassium carbonate, sodium bicarbonate), alkaline-earth metal carbonates (e.g., calcium carbonate), organic carbonates (e.g., guanidine carbonate), basic amino acids (arginine, lysine, histidine), and their polymers (poly arginine, poly lysine, etc.), organic amines, such as alkanolamines (e.g., monoethanolamine, diethanolamine, triethanolamine, aminomethyl propanol), ammonium hydroxide, and a mixture thereof.

Organic amines include, but are not limited to, those having one or two primary, secondary, or tertiary amine functions, and at least one linear or branched C1-C8 alkyl groups bearing at least one hydroxyl radical. Organic amines include cyclic amines and other cyclic compounds, saturated or unsaturated, having one or more nitrogen atoms within the ring, and mixtures thereof. The organic amines may be chosen from those having a pKb at 25° C. of less than 12, such as less than 10 or such as less than 6. It should be noted that this is the pKb corresponding to the function of highest basicity.

Organic amines may also be chosen from alkanolamines such as mono-, di- or trialkanolamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof.

Non-limiting examples of alkanolamines include monoethanolamine (also known as monoethanolamine or MEA), diethanolamine, triethanolamine, monoisopropanolamine, aminomethyl propoanol, diisopropanolamine, triisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol (aminomethyl propanol or AMP), tris(hydroxymethylamino)methane, tetrahydroxypropyl ethylenediamine, tromethamine, a mixture thereof, preferably triethanolamine and/or aminomethyl propanol. Other examples include but are not limited to: 1,3-diaminopropane, 1,3-diamino-2-propanol, spermine, and spermidine.

As non-limiting examples, the amino acids that may be used may be of natural or synthetic origin, in L, D, or racemic form, and comprise at least one acid function chosen from, for instance, carboxylic acid, sulfonic acid, phosphonic acid, and phosphoric acid functions. The amino acids may be in their neutral or ionic form. Polymeric forms are also useful, such as poly arginine, poly lysine, etc. Additional non-limiting examples include basic amino acids comprising an additional amine function optionally included in a ring or in a ureido function. In some cases, amino acids that may be used include, but are not limited to, aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, ornithine, citrulline, and valine.

In instances, the organic amines may be organic amines of heterocyclic type. Besides histidine that has already been mentioned in the amino acids, non-limiting mention may also be made of pyridine, piperidine, imidazole, 1,2,4-triazole, tetrazole, and benzimidazole. In some cases, the organic amines may be amino acid dipeptides. Amino acid dipeptides that may be used in the present disclosure include but not limited to carnosine, anserine, and baleine.

The organic amines may also be chosen from compounds comprising a guanidine function. Organic amines of this type include, besides arginine that has already been mentioned as an amino acid, creatine, creatinine, 1,1-dimethylguanidine, 1,1-diethylguanidine, glycocyamine, metformin, agmatine, N-amidinoalanine, 3-guanidinopropionic acid, 4-guanidinobutyric acid, and 2-([amino(imino)methyl]amino)ethane-1-sulfonic acid.

The organic amine may be in salt form. The term “organic amine salt,” as used herein, means organic or mineral salts of an organic amine as described above. As a non-limiting example, the organic salts may be chosen from the salts of organic acids, such as citrates, lactates, glycolates, gluconates, acetates, propionates, fumarates, oxalates and tartrates. In one embodiment, the organic salt is trisodium citrate. Further as a non-limiting example, the mineral salts may be chosen from hydrohalides (for example hydrochlorides), carbonates, hydrogen carbonates, sulfates, hydrogen phosphates, and phosphates. The ammonium salts that may be used according to the present disclosure may be chosen from the following acid salts: carbonate, bicarbonate. For instance, the salt is the carbonate, such as ammonium carbonate.

The alkali metal phosphates and carbonates that may be used are, for example, sodium phosphate, potassium phosphate, sodium carbonate, sodium bicarbonate, potassium carbonate, potassium bicarbonate, and their derivatives.

Alkaline agents may also be chosen from inorganic bases and hydroxide bases such as alkali metal hydroxides, alkaline-earth metal hydroxides, transition metal hydroxides, quaternary ammonium hydroxides, organic hydroxides, and mixtures thereof. Suitable examples are ammonium hydroxide, sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide and mixtures thereof.

In some instances, the at least one alkaline agent is chosen from aminomethyl propanol, sodium hydroxide, potassium hydroxide, lithium hydroxide, aminomethyl propanediol, triisopropanol amine, dimethylstearylamine, dimethyl/tallowamine, lysine, ornithine, arginine, monoethanolamine, triethanolamine, calcium hydroxide, calcium bicarbonate, and mixtures thereof. Furthermore, in some instances, the at least one alkaline agent is chosen from aminomethyl propanol, sodium hydroxide, lithium hydroxide, calcium hydroxide, monoethanolamine, triethanolamine, trisodiuim citrate, and mixtures thereof. Moreover, in some instances, the at least one alkaline agent is chosen from an alkanolamine, for example, an alkanolamine selected from monoethanolamine, diethanolamine, triethanolamine, aminomethyl propanol, tris(hydroxymethyl)aminomethane, tetra hydroxy propyl ethylenediamine, a mixture thereof, preferably triethanolamine, aminomethyl propanol, and a mixture thereof.

Water-Soluble Solvents

The term “water-soluble solvent” is interchangeable with the term “water-miscible solvent” and means a compound that at 25° C. and at atmospheric pressure (760 mmHg) has a solubility of at least 50% in water. In some cases, the water-soluble solvent has a solubility of at least 60%, 70%, 80%, or 90% in water at 25° C. and at atmospheric pressure (760 mmHg). Non-limiting examples of water-soluble solvents include, for example, glycerin, alcohols (for example, C₁₋₈ or C₁₋₄ alcohols), organic solvents, polyols, glycols, and a mixture thereof.

Non-limiting examples of water soluble solvents include monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylne glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

In some embodiments, the water-soluble solvent may be selected from one or more glycols, C₁₋₄ alcohols, glycerin, and a mixture thereof.

More exhaustive but non-limiting lists of components useful in the compositions disclosed herein are presented below.

Thickening Agents

Thickening agents may be to as “thickeners” or “viscosity modifying agents.” Many thickening agents are water-soluble, and increase the viscosity of water or form an aqueous gel when dispersed/dissolved in water. The aqueous solution may be heated and cooled, or neutralized, for forming the gel, if necessary. The thickening agent may be dispersed/dissolved in an aqueous solvent that is soluble in water, e.g., ethyl alcohol when it is dispersed/dissolved in water.

Non-limiting examples of thickening agents include xanthan gum, guar gum, biosaccharide gum, cellulose, acacia Seneca gum, sclerotium gum, agarose, pechtin, gellan gum, hyaluronic acid. In some instances, the one or more thickening agents may include polymeric thickening agents, for example, those selected from the group consisting of ammonium polyacryloyldimethyl taurate, ammonium acryloyldimethyltaurate/VP copolymer, sodium polyacrylate, acrylates copolymers, polyacrylamide, carbomer, acrylates/C10-30 alkyl acrylate crosspolymer, and acrylamide/sodium acryloyldimethyltaurate copolymer

In some instances, the thickening agent(s) are selected from carboxylic acid polymers (e.g., carbomer), crosslinked polyacrylate polymers, polyacrylamide polymers, polysaccharides, gums, and a mixture thereof. A more detailed description of various thickening agents is provided below.

(a) Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include Ultrez® 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as Carbopol® 1342, Carbopol® 1382, Pemulen TR-1, and Pemulen TR-2, from B.F. Goodrich. In other words, examples of carboxylic acid polymer thickeners useful herein are those selected from carbomers, acrylates/C10-C30 alkyl acrylate crosspolymers, and mixtures thereof.

(b) Crosslinked Polyacrylate Polymers

The compositions of the present disclosure can optionally contain crosslinked polyacrylate polymers useful as thickeners or gelling agents including both cationic and nonionic polymers.

(c) Polyacrylamide Polymers

The compositions of the present disclosure can optionally contain polyacrylamide polymers, especially polyacrylamide polymers including substituted branched or unbranched polymers. Among these polyacrylamide polymers is the polymer given the CTFA designation polyacrylamide and isoparaffin and laureth-7, available under the Tradename Sepigel 305 from Seppic Corporation.

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc.

The compositions may also contain thickening and texturizing gels of the type as exemplified by the product range called Lubrajel® from United Guardian. These gels have moisturizing, viscosifying, stabilizing properties.

(d) Polysaccharides

A wide variety of polysaccharides can be useful herein. “Polysaccharides” refer to gelling agents that contain a backbone of repeating sugar (i.e., carbohydrate) units. Nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation.

Other useful polysaccharides include scleroglucans comprising a linear chain of (1-3) linked glucose units with a (1-6) linked glucose every three units, a commercially available example of which is Clearogel™. CS11 from Michel Mercier Products Inc.

(e) Gums

Other thickening and gelling agents useful herein include materials which are primarily derived from natural sources. Nonlimiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, biosacharide gum, and mixtures thereof.

Additional examples of water-soluble thickeners include water-soluble natural polymers, water-soluble synthetic polymers, clay minerals and silicic anhydride. Non-limiting examples of water-soluble natural polymers include gum arabic, tragacanth gum, karaya gum, guar gum, gellan gum, tara gum, locust bean gum, tamarind gum, sodium alginate, alginic acid propyleneglycol ester, carrageenan, farcelluran, agar, high-methoxy pectin, low-methoxy pectin, xanthine, chitosan, starch (for example starch derived from corn, potato, wheat, rice, sweet potato and tapioca, a-starch, soluble starch), fermentation polysaccharide (for example, xanthan gum, pullulan, carciran, dextran), acidic hetero-polysaccharide derived from callus of plants belonging to Polyantes sp. (for example, tuberous polysaccharide), proteins (for example, sodium casein, gelatin, albumin), chondroitin sulfate, and hyaluronic acid.

Non-limiting examples of water-soluble synthetic polymers include polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (for example, carboxymethyl cellulose, methyl cellulose, methylhydroxypropyl cellulose, hydroxyethyl cellulose, hydroxypropyl cellulose, cellulose sulfate sodium salt), and starch derivatives (for example, starch oxide, dialdehyde starch, dextrin, British gum, acetyl starch, starch phosphate, carboxymethyl starch, hydroxyethyl starch, hydroxypropyl starch).

Non-Silicone Fatty Compounds

The term “non-silicone fatty compound” means a fatty compound that does not containing any silicon atoms (Si). Non-limiting examples of non-silicone fatty compounds include oils, mineral oil, fatty alcohols, fatty acids, fatty alcohol derivatives, fatty acid derivatives (such as alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof. Non-limiting examples of the fatty alcohols, fatty acids, fatty alcohol derivatives, and fatty acid derivatives are found in International Cosmetic Ingredient Dictionary, Sixteenth Edition, 2016, which is incorporated by reference herein in its entirety.

Fatty alcohols useful herein include those having from about 10 to about 30 carbon atoms, from about 12 to about 22 carbon atoms, and from about 16 to about 22 carbon atoms. These fatty alcohols can be straight or branched chain alcohols and can be saturated or unsaturated. Nonlimiting examples of fatty alcohols include decyl alcohol, undecyl alcohol, dodecyl, myristyl, cetyl alcohol, stearyl alcohol, isostearyl alcohol, isocetyl alcohol, behenyl alcohol, linalool, oleyl alcohol, cholesterol, cis4-t-butylcyclohexanol, myricyl alcohol and a mixture thereof. In some cases, the fatty alcohols are those selected from the group consisting of cetyl alcohol, stearyl alcohol, isostearyl alcohol, oleyl alcohol, and a mixture thereof.

Fatty acids useful herein include those having from about 10 to about 30 carbon atoms, from about 12 to about 22 carbon atoms, and from about 16 to about 22 carbon atoms. These fatty acids can be straight or branched chain acids and can be saturated or unsaturated. Also included are diacids, triacids, and other multiple acids which meet the carbon number requirement herein. Also included herein are salts of these fatty acids. Nonlimiting examples of fatty acids include lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

Fatty alcohol derivatives include alkyl ethers of fatty alcohols, alkoxylated fatty alcohols, alkyl ethers of alkoxylated fatty alcohols, esters of fatty alcohols and a mixture thereof. Nonlimiting examples of fatty alcohol derivatives include materials such as methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

Non-limiting examples of polyglycerol esters of fatty acids include those of the following formula:

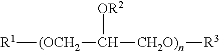

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀. For example, nonionic polyglycerol esters of fatty acids include polyglyceryl-5 laurate,

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, and a mixture thereof. Nonlimiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

In some cases, the one or more non-silicone fatty compounds may be one or more high melting point fatty compounds. A high melting point fatty compound is a fatty compound having a melting point of 25° C. Even higher melting point fatty compounds may also be used, for example, fatty compounds having a melting point of 40° C. or higher, 45° C. or higher, 50° C. or higher. The high melting point fatty compound may be selected from the group consisting of fatty alcohols, fatty acids, fatty alcohol derivatives, fatty acid derivatives, and mixtures thereof. Nonlimiting examples of the high melting point compounds are found in the International Cosmetic Ingredient Dictionary, Sixteenth Edition, 2016, which is incorporated by reference herein in its entirety. The fatty alcohols useful herein are those having from about 14 to about 30 carbon atoms, preferably from about 16 to about 22 carbon atoms. These fatty alcohols are saturated and can be straight or branched chain alcohols. Non-limiting examples of high melting point fatty compounds include fatty alcohols such as, for example, cetyl alcohol (having a melting point of about 56° C.), stearyl alcohol (having a melting point of about 58-59° C.), behenyl alcohol (having a melting point of about 71° C.), and mixtures thereof. These compounds are known to have the above melting point. However, they often have lower melting points when supplied, since such supplied products are often mixtures of fatty alcohols having alkyl chain length distribution in which the main alkyl chain is cetyl, stearyl or behenyl group. In the present application, more preferred fatty alcohols are cetyl alcohol, stearyl alcohol and mixtures thereof.

In some instances, the non-silicone fatty compounds include one or more waxes. The waxes generally have a melting point of from 35-120° C., at atmospheric pressure. Non-limiting examples of waxes in this category include for example, synthetic wax, ceresin, paraffin, ozokerite, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, sunflower seed wax (Helianthus annuus), acacia decurrents flower wax, or a mixture thereof.

In one embodiment, the personal care composition includes 10-30% of a combination of waxes. Mention may be made, among the waxes capable of being used as non-silicone fatty compounds, of animal waxes, such as beeswax; vegetable waxes, such as sunflower seed (Helianthus annuus), carnauba, candelilla, ouricury or japan wax or cork fibre or sugarcane waxes; mineral waxes, for example paraffin or lignite wax or microcrystalline waxes or ozokerites; synthetic waxes, including polyethylene waxes, and waxes obtained by the Fischer-Tropsch synthesis.

In some instance, the non-silicone fatty compounds include one or more non-silicone oils. The term “oil” as used herein describes any material which is substantially insoluble in water. Suitable non-silicone oils include, but are not limited to, natural oils, such as coconut oil; hydrocarbons, such as mineral oil and hydrogenated polyisobutene; fatty alcohols, such as octyldodecanol; esters, such as C₁₂-C₁₅ alkyl benzoate; diesters, such as propylene dipelarganate; and triesters, such as glyceryl trioctanoate. Suitable low viscosity oils have a viscosity of 5-100 mPas at 25° C., and are generally esters having the structure RCO—OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or combinations thereof. The high viscosity oils generally have a viscosity of 200-1,000,000, or 100,000-250,000, mPas at 25° C. Such oils include castor oil, lanolin and lanolin derivatives, triisocetyl citrate, sorbitan sesquioleate, C₁₀-C₁₈ triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

Mineral oils, such as liquid paraffin or liquid petroleum, or animal oils, such as perhydrosqualene or arara oil, or alternatively of vegetable oils, such as sweet almond, calophyllum, palm, castor, avocado, jojoba, olive or cereal germ oil, may be utilized. It is also possible to use esters of these oils, e.g., jojoba esters. Also useful are esters of lanolic acid, of oleic acid, of lauric acid, of stearic acid or of myristic acid; esters of alcohols, such as oleyl alcohol, linoleyl or linolenyl alcohol, isostearyl alcohol or octyldodecanol; and/or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

Emulsifiers

Emulsifiers are well known in the art and include amphoteric, anionic, cationic or nonionic emulsifiers, used alone or as a mixture, and optionally with a co-emulsifier. The emulsifiers are chosen in an appropriate manner according to the emulsion to be obtained.

For W/O emulsions, examples of emulsifiers that may be mentioned include dimethicone copolyols, such as the mixture of cyclomethicone and dimethicone copolyol sold under the trade name DC 5225 C by the company Dow Corning, and alkyl dimethicone copolyols such as the lauryl dimethicone copolyol sold under the name Dow Corning 5200 Formulation Aid by the company Dow Corning, and the cetyl dimethicone copolyol sold under the name Abil EM 90™ by the company Goldschmidt.

For O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof.

In some instance, the one or more emulsifiers include an oganosiloxane emulsifier, including crosslinked organosiloxane emulsifiers. For example, the compositions may comprise one or more crosslinked organosiloxane emulsifier selected from the group consisting of dimethicone/dimethicone PEG/PPG 15 crosspolymer, dimethicone PEG-10 crosspolymer, dimethicone PEG-10/15 crosspolymer, dimethicone PEG-15 crosspolymer, dimethicone polyglycerin-3 crosspolymer, dimethicone PPG-20 crosspolymer, dimethiconol/methylsilanol/silicate crosspolymer; dimethiconol/silicate crosspolymer, lauryl dimethicone PEG-15 crosspolymer, lauryl dimethicone polyglycerin-3 crosspolymer, PEG-8 dimethicone polysorbate-20 crosspolymer, PEG-10 dimethicone/vinyl dimethicone crosspolymer, PEG-10 lauryl dimethicone crosspolymer, PEG-15/lauryl dimethicone crosspolymer, PEG-15 laurylpolydimethylsiloxyethyl crosspolymer.

In another embodiment, the compositions include one or more linear organosiloxane emulsifier selected from the group consisting of cyclotetrasiloxane (and) cyclopentasiloxane (and) PEG/PPG-18 dimethicone, cyclopentasiloxane (and) PEG/PPG-18/18 dimethicone; PEG/PPG-18/18 dimethicone; lauryl PEG/PPG-18/18 methicone; cetyl PEG/PPG-14/14 dimethicone; bis-cetyl PEG/PPG-14/14 dimethicone; cetyl PEG/PPG-10/1 dimethicone; PEG-11 methyl ether dimethicone; PEG/PPG-20/22 butyl ether dimethicone; PEG-9 dimethicone; PEG-3 dimethicone; PEG-9 methyl ether dimethicone; PEG-10 dimethicone; lauryl PEG-9 polydimethylsiloxyethyl dimethicone.

Usable oxyalkylenated organosiloxane emulsifier include the following:

An oxyalkylenated organosiloxane emulsifier having the general formula:

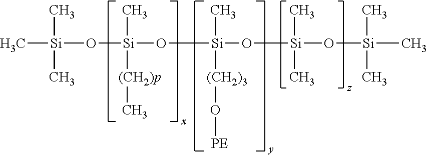

wherein p is 0-40 (the range including all numbers between and subranges such as 2, 3, 4, 13, 14, 15, 16, 17, 18, etc.), and PE is (—C₂H₄O)ₐ-(—C₃H₆O)_{b}—H wherein a is 0-25, b is 0-25 with the proviso that both a and b cannot be 0 simultaneously, x, y, and z are each independently ranging from 0 to 1 million with the proviso that x and y cannot be 0 simultaneously. In some cases, x, y, z, a, and b are such that the molecular weight of the polymer ranges from about 5,000 to about 500,000, from about 10,000 to 100,000, or is about 50,000, and the polymer is generically referred to as dimethicone copolyol. In some instances, p is such that the long chain alkyl is cetyl or lauryl, and the the compound is called, generically, cetyl dimethicone copolyol or lauryl dimethicone copolyol respectively. In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or ether, such as methyl ether, ethyl ether, propyl ether, butyl ether, and the like.

An oxyalkylenated organosiloxane emulsifier having the general formula:

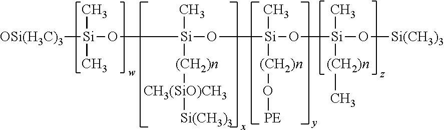

wherein each n is independently 0-100 with the proviso that there must be at least one PE radical. In some instances, where each n independently ranges from about 2 to 30, and PE (—C₂H₄O)ₐ-(—C₃H₆O)_{b}—H wherein a is 0-25, b is 0-25 with the proviso that both a and b cannot simultaneously be 0; and wherein w, x, y, and z are each independently 0 to 1,000,000 with the proviso that there is at least one PE. In some embodiments the organosiloxane emulsifier is lauryl PEG-9 Polydimethylsiloxyethyl Dimethicone. Oxyalkylenated organosiloxane emulsifiers disclosed in U.S. Pat. No. 9,095,543 are useful in the instant compositions. U.S. Pat. No. 9,095,543 is incorporated herein by reference in its entirety.

Further examples of organosiloxane emulsifiers include those having C.T.F.A. names Bis-Butyldimethicone Polyglyceryl-3; Bis-PEG/PPG-14/14 Dimethicone; Bis-butyldimethicone Polyglyceryl-3; Bis-isobutyl PEG/PPG-10/7 Dimethicone copolymer; Bis-PEG/PPG-18/6 Dimethicone; Bis-PEG/PPG-20/20 Dimethicone; Bis-PEG/PPG-16/16 PEG/PPG-16/16 Dimethicone; Bis(PPG-7 Undeceneth-21-Dimethicone; Cetyl Dimethicone PEG-7 Acetate; Cetyl PEG-8 Dimethicone; Cetyl PEG/PPG-15/16 Butyl Ether Dimethicone; Cetyl PEG/PPG-15/15 Butyl Ether Dimethicone; Cetyl PEG/PPG-7/3 Dimethicone; Cetyl PEG/PPG-10/1 Dimethicone; Dimethicone PEG-15 Acetate; Dimethicone PEG-7 Cocoate; Dimethicone PEG-7 Phosphate; Dimethicone PEG-10 Phosphate; Dimethicone PEG/PPG-7/4 Phosphate; Dimethicone PEG/PPG-12/4 Phosphate; Dimethicone PEG-7 Undecylenate; Lauryl Dimethicone PEG-10 Phosphate; Isopolyglyceryl-3 Dimethicone; Isopolyglyceryl-3 Dimethiconol; Isostearyl Carboxyldecyl PEG-8 Dimethicone; Lauryl Methicone PEG-10 Phosphate; Lauryl PEG-8 Dimethicone; Lauryl PEG-10 Methyl Ether Dimethicone; Lauryl PEG/PPG-18/18 Methicone; PEG-6 Methyl Ether Dimethicone; PEG-7 Methyl Ether Dimethicone; PEG-9 Methyl Ether Dimethicone; PEG-10 Methyl Ether Dimethicone; PEG-11 Methyl Ether Dimethicone; PEG-11 Methyl Ether Dimethicone; PEG-32 Methyl Ether Dimethicone; PEG-PEG/PPG-28/21 Acetate Dimethicone; PEG/PPG-22/22 Butyl Ether Dimethicone; PEG/PPG-23/23 Butyl Ether Dimethicone; PEG/PPG-24/18 Butyl Ether Dimethicone; PEG/PPG-3/10 Dimethicone; PEG/PPG-4/12 Dimethicone; PEG/PPG-6/11 Dimethicone; PEG/PPG-8/14 Dimethicone; PEG/PPG-12/16 Dimethicone; PEG/PPG-12/18 Dimethicone; PEG/PPG-14/4 Dimethicone; PEG/PPG-15/5 Dimethicone; PEG/PPG-15/15 Dimethicone; PEG/PPG-16/2 Dimethicone; PEG/PPG-16/8 Dimethicone; PEG/PPG-17/18 Dimethicone; PEG/PPG-18/12 Dimethicone; PEG/PPG-19/19 Dimethicone; PEG/PPG-20/6 Dimethicone; PEG/PPG-20/15 Dimethicone; PEG/PPG-20/20 Dimethicone; PEG/PPG-20/29 Dimethicone; PEG/PPG-22/23 Dimethicone; PEG/PPG-22/24 Dimethicone; PEG/PPG-25/25 Dimethicone; PEG/PPG-27/27 Dimethicone; PEG/PPG-30/10 Dimethicone; PEG/PPG-10/3 Oleyl Ether Dimethicone; PEG-8 trisiloxane; Polyglyceryl-3 Polydimethylsiloxyethyl Dimethicone; PPG-12 Butyl Ether Dimethicone; Silicone Quaternium-17; TEA-Dimethicone PEG-7 Phosphate; or mixtures thereof.

Further examples of commercial linear organosiloxane emulsifiers are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

Also suitable are various types of fully or partially crosslinked oxyalkylenated organosiloxane emulsifiers. They may be elastomeric or non-elastomeric. They are sometimes referred to as “emulsifying elastomers” because of they have both elastomeric and emulsifying properties.

Polyoxyalkylenated silicone elastomers that may be used in at least one embodiment include those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers include dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

Further examples of crosslinked organosiloxane emulsifiers include, but are not limited to Dimethicone/dimethicone PEG/PPG 15 crosspolymer; Dimethicone PEG-10 crosspolymer; Dimethicone PEG-10/15 Crosspolymer; Dimethicone PEG-15 Crosspolymer; Dimethicone Polyglycerin-3 Crosspolymer; Dimethicone PPG-20 Crosspolymer; Dimethiconol/Methylsilanol/Silicate Crosspolymer; Dimethiconol/Silicate Crosspolymer; Lauryl Dimethicone PEG-15 Crosspolymer; Lauryl Dimethicone Polyglycerin-3 Crosspolymer; PEG-8 Dimethicone Polysorbate-20 Crosspolymer; PEG-10 Dimethicone/Vinyl Dimethicone Crosspolymer; PEG-10 Lauryl Dimethicone Crosspolymer; PEG-15/Lauryl Dimethicone Crosspolymer; and PEG-15 Laurylpolydimethylsiloxyethyl Crosspolymer.

Implementation of the present disclosure is provided by way of the following examples. The examples serve to illustrate the technology without being limiting in nature.

| | | Solutions | | | | Gels | | | Emulsions | | |
|---|---|---|---|---|---|---|---|---|---|---|---|
| | | INCI US | | | | | | | | | |
| Alkaline Compositions | | A | B | C | D | E | F | G | H | I | J |
| Alkaline | SODIUM SILICATE | 3.7 | | | | 3.7 | 3.7 | 3.7 | | | |
| Agents | POTASSIUM HYDROXIDE | | | | 0.1 | | | | | | |
| | ARGININE | | 5 | | | | | | | | |
| | SODIUM BICARBONATE | | | 5 | | | | | | | |
| | GUANIDINE CARBONATE | | | | | | | | 4 | | |
| | TRIETHANOLAMINE | | | | | | | | | 5 | |
| | ETHANOLAMINE | | | | | | | | | | 4 |
| Water-Soluble | GLYCERIN | 10 | 10 | 10 | 10 | 10 | 10 | 10 | | | |
| Solvents | | | | | | | | | | | |
| Non-Silicone | MINERAL OIL | | | | | | | | 63.5 | 63.5 | 63.5 |
| Fatty Compounds | ISOHEXADECANE | | | | | 1.4 | 1.7 | | | | |
| Emulsifiers | DECYL GLUCOSIDE | | | | | | | | 1.1 | 1.1 | 1.1 |
| | CETETH-2 | | | | | | | | 1 | 1 | 1 |
| | SORBITAN OLEATE | | | | | 0.2 | 0.2 | | | | |
| | POLYSORBATE 80 | | | | | 0.5 | 0.6 | | | | |
| Thickening Agents | CETYL | | | | | | | | 0.6 | 0.6 | 0.6 |
| | HYDROXYETHYLCELLULOSE | | | | | | | | | | |
| | ACRYLAMIDE/SODIUM | | | | | 2.6 | 3.2 | | | | |
| | ACRYLOYLDIMETHYL-TAURATE | | | | | | | | | | |
| | COPOLYMER | | | | | | | | | | |
| | HYDROXYETHYL | | | | | | | 4.5 | | | |
| | ACRYLATE/SODIUM | | | | | | | | | | |
| | ACRYLOYLDIMETHYL TAURATE | | | | | | | | | | |
| | COPOLYMER | | | | | | | | | | |
| Misc. | PRESERVATIVES, CHELATING | ≤3 | ≤3 | ≤3 | ≤3 | ≤3 | ≤3 | ≤3 | ≤3 | ≤3 | ≤3 |
| | AGENTS, VITAMINS, | | | | | | | | | | |
| | FRAGANCE, COLORANTS, pH | | | | | | | | | | |
| | ADJUSTERS, ETC. | | | | | | | | | | |
| pH | | 12 | 12 | 12 | 12 | 12 | 12 | 12 | 12 | 12 | 12 |
| | WATER | 86.3 | 85 | 83 | 90 | 82 | 81 | 81 | 30 | 30 | 30 |

### Example 2

### Acidic Compositions

| | | Solution | Gel | Emulsion |
|---|---|---|---|---|
| | | INCI US | | |
| Acidic Compositions | | H | I | J |
| Acids | LACTIC ACID | 10 | 10 | 10 |
| | SALICYLIC ACID | 0.5 | 0.5 | 0.5 |
| Water-Soluble | GLYCERIN | | | 7 |
| Solvent | ALCOHOL DENAT. | 10.7 | 12 | 10.7 |
| Thickening | POLYACRYLATE CROSSPOLYMER-6 | | 2 | |
| Agent | HYDROXYETHYLCELLULOSE | | | 0.2 |
| Silicone | DIMETHICONE | | | 7.7 |
| | POLYMETHYLSILSESQUIOXANE | | | 2 |
| Silicone | PEG-9 POLYDIMETHYLSILOXYETHYL | | | 1 |
| (surfactant) | DIMETHICONE | | | |
| Silicone | DIMETHICONE/PEG-10/15 | | | 1.3 |
| (film-forming) | CROSSPOLYMER | | | |
| | ACRYLATES/STEARYL | | | 1 |
| | ACRYLATE/DIMETHICONE | | | |
| | METHACRYLATE COPOLYMER | | | |
| Filler | SILICA AND/OR BORON NITRIDE | | | 2.7 |
| Fatty | SYNTHETIC WAX | | | 4 |
| Compound | ISONONYL ISONONANOATE | | | 4.5 |
| | DICAPRYLYL CARBONATE | | | 4.5 |
| Skin Active | SODIUM HYALURONATE | | | 0.1 |
| | PRESERVATIVES, CHELATING AGENTS, | ≤3 | ≤3 | ≤3 |
| | VITAMINS, FRAGRANCE, COLORANTS, | | | |
| | pH ADJUSTERS, ETC. | | | |
| pH | | 3.5 | 3.5 | 3.5 |
| | WATER | 77.1 | 72.5 | 41.4 |

### Example 3

### In Vitro Performance

Various compositions (alkaline versus acidic) and treatment protocols were tested and compared to determine which compositions and protocols produced the greatest degree of lipid disorganization, which is a common method for estimating the desquamation efficacy. All DSC experiments were performed on isolated human stratum corneum (SC). The SC was prepared using a standard protocol. Human abdominal skin remaining from plastic surgery was obtained in frozen form. Subcutaneous fat was removed and the dermis was separated from epidermis by plunging the skin into hot water (56° C.) for 1-2 min. The remaining viable epidermis was removed by tryptic digestion at 37° C. overnight. The SC was dried and stored at room temperature and ambient humidity. Samples of stratum corneum were equilibrated at 75% relative humidity (RH), at room temperature (RT) at least overnight before testing. The following five protocols were tested:
1. Positive Control: A solution of 70% glycolic acid (GA) was tested as a positive control.
2. Alkaline Treatment Only: Composition A of Example 1 (containing sodium silicate as the alkaline agent) was used for the alkaline treatment.
3. Acidic Treatment Only: Composition H of Example 2 (containing lactic acid and salicylic acid) was used for the acidic treatment.
4. Acidic Treatment Followed by Alkaline Treatment: Composition H of Example 2 (containing lactic acid and salicylic acid) was used for the acidic treatment. This acidic treatment was followed by treatment with Composition A of Example 1 (containing sodium silicate as the alkaline agent).
5. Alkaline Treatment Followed by Acidic Treatment: Composition A of Example 1 (containing sodium silicate as the alkaline agent) was used for the alkaline treatment. This alkaline treatment was followed by treatment with Composition H of Example 2 (containing lactic acid and salicylic acid).

0.1 gram of each of a test composition was applied to a sample of stratum corneum. The test composition was allowed to remain on the stratum corneum for 15 minutes at 75% RH, at RT, after which the surface of the stratum corneum was wiped clean, rinsed with deionized water and tab dry with tissue paper. For samples of protocol 4 and 5, they were subsequently treated with an alkaline treatment and an acidic treatment, respectively, for 15 minutes at 75% RH, at RT, after which the surface of the stratum corneum was wiped clean and rinsed with deionized water. After rinsing, samples were allowed to dry at 75% RH, at RT overnight.

The intercellular lipids in stratum corneum are organized in highly ordered multilayers where lipid aliphatic chains are predominantly arranged in a crystalline structure. This tight packing of lipid molecules ensures the barrier function of the stratum corneum. Any treatment that reduces lipid ordering will potentially impair the barrier function, thus promoting the penetration of topically applied substances through the SC. The degree of lipid disorganization following treatment can be conveniently monitored by the shifts of the melting transition temperatures measured by Differential Scanning Calorimetry (DSC). The thermogramme of the untreated stratum corneum is characterized by 4 phase transitions:

T1—around 40° C. corresponding to the change of lipid chain packing from orthorhombic to hexagonal;

T2—around 72° C. corresponding in the melting of alkyl chains;

T3—around 85° C. corresponding to the disturbance of the lamellar arrangement of lipids and loss of ordering at the level of polar heads (this peak is also often attributed to corneocyte-bound lipids);

T4—above 110° C. (irreversible) corresponding to the denaturation of proteins¹.

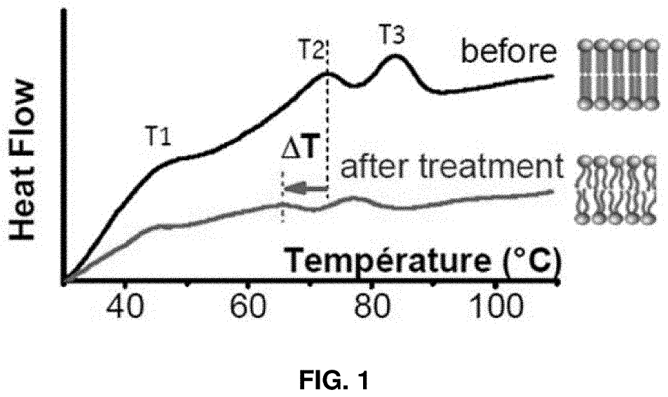

The endothermic peaks corresponding to transitions T1, T2 and T3 for a typical SC sample are shown in FIG. 1.

Shifts in the lipid phase transition temperatures of T2 and T3 following treatment are interpreted as evidence of product penetration into the SC, interaction with intercellular lipids and disordering (fluidizing) effect on their crystal organization. Lipid disordering on the molecular level is translated into a deterioration of the barrier properties of the SC at the tissue level. A decrease of 2° C. is considered significant. In addition, if the shift of melting temperature is associated with a drop of transition enthalpy (the area under the peak), this can be interpreted as a partial loss (extraction) of the lipids during treatment. The measurements were performed using DSC Q200 (TA Instruments) using Tzero aluminium hermetic pans and heating from 20° to 120° C. at a rate of 5° C./min. An empty pan was used as a reference.

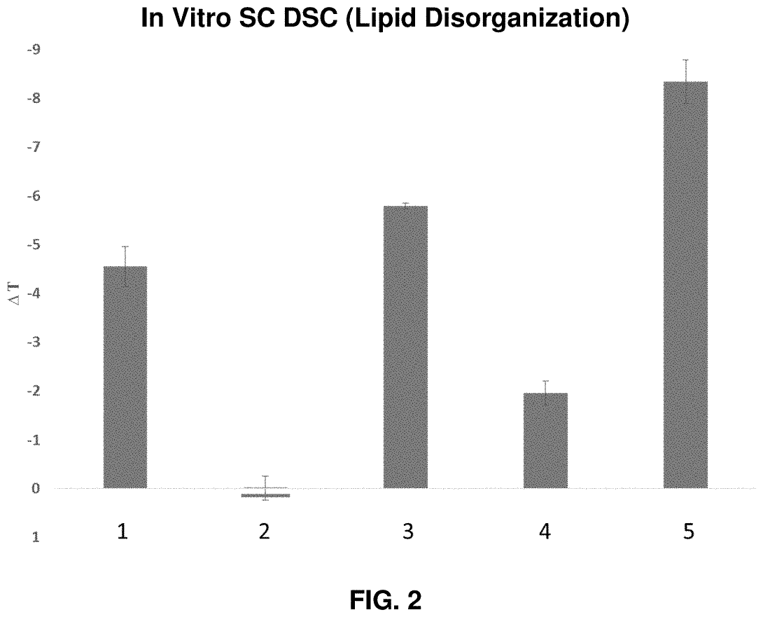

The maxima of the endothermic peaks corresponding to lipid phase transitions are determined using dedicated software (TA Universal Analysis). The peak position and the transition enthalpy are determined with respect to a manually defined linear baseline. Specific enthalpy is calculated using the hydrated (75% RH) SC mass before treatment. The shifts of the melting peak positions (ΔTm(T2) and ΔTm(T3)) are calculated with respect to reference thermogrammes obtained with untreated or vehicle-treated samples. For simplicity, an effective shift is calculated as a linear combination of the measured shifts for the T2 and T3 transitions for each treatment: ΔTeffective=0.7·ΔTm(T2)+0.3ΔTm(T3). Higher weight is given to the T2 transition as it is our primary criterion of lipid perturbation with well-established interpretation; at the same time, the T3 contribution is also included. The results are reported in the table below and graphically presented in FIG. 2.

| Protocol | | Compositions | ΔT | Std |
|---|---|---|---|---|
| 1 | Positive Control | 70% GA | −4.6 | 0.4 |
| 2 | Alkaline Treatment | Composition A of Example 1 | −0.004 | 0.2 |
| | Only | (pH 12) | | |
| 3 | Acidic Treatment Only | Composition H of Example 2 | −5.8 | 0.1 |
| 4 | Acidic Treatment | Composition H of Example 2 | −2.0 | 0.2 |
| | Followed by Alkaline | Followed by Composition A | | |
| | Treatment | of Example 1 | | |
| 5 | Alkaline Treatment | Composition A of Example 1 | −8.3 | 0.4 |
| | Followed by Acidic | Followed by Composition H | | |
| | Treatment | of Example 2 | | |

Treatment with only the alkaline composition (Protocol 2) had basically no influence on desquamation. As expected, the acidic composition provided an appreciable degree of desquamation. Treating the stratum corneum with the acidic composition first followed by the alkaline composition, however, surprisingly provided worse results than treating the skin with only the acidic composition. More surprisingly, however, is the finding that reversing the order and treating the stratum corneum first with an alkaline composition followed by treatment with an acidic composition provides a significant improvement in the desquamation of the stratum corneum. Protocol 5 (treating the skin with an alkaline composition followed by treatment with an acidic composition) provided a ΔT of −8.3, which is significantly higher than the ΔT for both the positive control (−4.6) and the acidic treatment alone (−5.8).

### Example 4

### In Vitro Performance

Testing was carried out using a variety of different alkaline agents in the formulation of alkaline compositions. These alkaline compositions were tested on samples of stratum corneum in the same manner described above in Example 3. Nine different protocols were tested.
1. Positive Control: A solution of 70% glycolic acid (GA) was tested as a positive control.
2. Acidic Treatment Only: Composition H of Example 2 (containing lactic acid and salicylic acid) was used for the acidic treatment.
3. Alkaline Treatment Only: Composition B of Example 1 (containing arginine was an alkaline agent) was used for the alkaline treatment.
4. Alkaline Treatment Followed by Acidic Treatment: Composition B of Example 1 (containing arginine as the alkaline agent) was used for the alkaline treatment. This alkaline treatment was followed by treatment with Composition H of Example 2 (containing lactic acid and salicylic acid).
5. Acidic Treatment Followed by Alkaline Treatment: Composition H of Example 2 (containing lactic acid and salicylic acid) was used for the acidic treatment. This acidic treatment was followed by treatment with Composition B of Example 1 (containing arginine as the alkaline agent).
6. Alkaline Treatment Only: Composition C of Example 1 (containing sodium bicarbonate was an alkaline agent) was used for the alkaline treatment.
7. Alkaline Treatment Followed by Acidic Treatment: Composition C of Example 1 (containing sodium bicarbonate as the alkaline agent) was used for the alkaline treatment. This alkaline treatment was followed by treatment with Composition H of Example 2 (containing lactic acid and salicylic acid).
8. Acidic Treatment Followed by Alkaline Treatment: Composition H of Example 2 (containing lactic acid and salicylic acid) was used for the acidic treatment. This acidic treatment was followed by treatment with Composition C of Example 1 (containing sodium bicarbonate as the alkaline agent).
9. Alkaline Treatment Followed by Acidic Treatment: Composition A of Example 1 (containing sodium silicate as the alkaline agent) was used for the alkaline treatment. This alkaline treatment was followed by treatment with Composition H of Example 2 (containing lactic acid and salicylic acid).

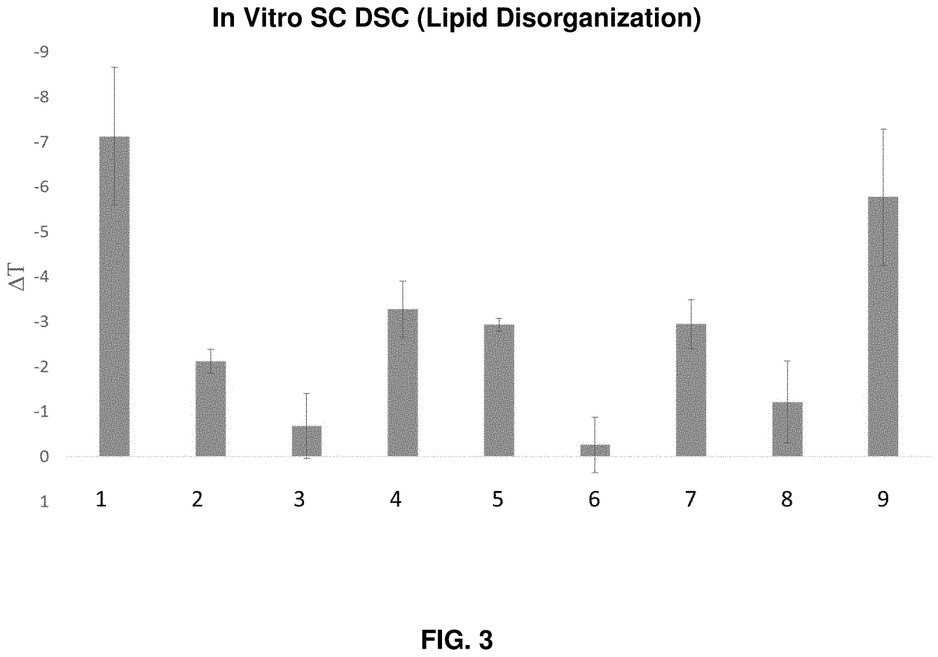

The results are reported in the table below and graphically presented in FIG. 3.

| Protocol | | Compositions | ΔT | Std |
|---|---|---|---|---|
| 1 | Positive Control | 70% GA | −7.1 | 1.5 |
| 2 | Acidic Treatment Only | Composition H of Example 2 | −2.1 | 0.3 |
| 3 | Alkaline Treatment Only | Composition B of Example 1 | −0.7 | 0.7 |
| 4 | Alkaline Treatment | Composition B of Example 1 | −3.3 | 0.6 |
| | Followed by Acidic | Followed by Composition H | | |
| | Treatment | of Example 2 | | |
| 5 | Acidic Treatment | Composition H of Example 2 | −2.9 | 0.1 |
| | Followed by Alkaline | Followed by Composition B | | |
| | Treatment | of Example 1 | | |
| 6 | Alkaline Treatment Only | Composition C of Example 1 | −0.3 | 0.6 |
| 7 | Alkaline Treatment | Composition C of Example 1 | −2.9 | 0.5 |
| | Followed by Acidic | Followed by Composition H | | |
| | Treatment | of Example 2 | | |
| 8 | Acidic Treatment | Composition H of Example 2 | −1.2 | 0.9 |
| | Followed by Alkaline | Followed by Composition C | | |
| | Treatment | of Example 1 | | |
| 9 | Alkaline Treatment | Composition A of Example 1 | −5.8 | 1.5 |
| | Followed by Acidic | Followed by Composition H | | |
| | Treatment | of Example 2 | | |

Treatment with only the alkaline compositions had basically no influence on desquamation. As expected, the acidic composition provided an appreciable degree of desquamation. Treating the stratum corneum with the acidic composition first followed by an alkaline composition showed little or no improvement over treatment with the acidic treatment alone; and in some cases worse results than acidic treatment alone (see protocol 8). Surprisingly, reversing the order and treating the stratum corneum first with an alkaline composition followed by treatment with an acidic composition provides a significant improvement in the desquamation of the stratum corneum.

### Example 5

### In Vivo Performance

In vivo testing was carried out on the forearms of volunteers to determine the desquamation efficacy of various compositions and protocols. The following six protocols were tested.
1. Placebo Control: A DI water was tested as a placebo control.
2. Acidic Treatment Only: Composition H of Example 2 (containing lactic acid and salicylic acid) was used for the acidic treatment.
3. Alkaline Treatment Followed by Acidic Treatment: Composition B of Example 1 (containing arginine as the alkaline agent) was used for the alkaline treatment. This alkaline treatment was followed by treatment with Composition H of Example 2 (containing lactic acid and salicylic acid).
4. Alkaline Treatment Followed by Acidic Treatment: Composition C of Example 1 (containing sodium bicarbonate as the alkaline agent) was used for the alkaline treatment. This alkaline treatment was followed by treatment with Composition H of Example 2 (containing lactic acid and salicylic acid).
5. Alkaline Treatment Followed by Acidic Treatment: Composition D of Example 1 (containing potassium hydroxide as the alkaline agent) was used for the alkaline treatment. This alkaline treatment was followed by treatment with Composition H of Example 2 (containing lactic acid and salicylic acid).
6. Alkaline Treatment Followed by Acidic Treatment: Composition A of Example 1 (containing sodium silicate as the alkaline agent) was used for the alkaline treatment. This alkaline treatment was followed by treatment with Composition H of Example 2 (containing lactic acid and salicylic acid).

The forearms of volunteers were rinsed with deionized water and dried for 3 minutes. 0.1 gram of test composition was applied to three designated regions on the forearm using a saturated cotton pad. The test composition was allowed to remain on the forearm for 15 minutes, after which the forearm was rinsed and patted dry with a tissue. After drying with the tissue, the forearm was allowed to continue to air dry for 3 minutes. For protocols 3-6, step 1 was performed using the designated alkaline compositions. The alkaline compositions were applied to the same three designated regions on the forearm using a saturated cotton pad. The test composition was allowed to remain on the forearm for 15 minutes, after which the forearm was rinsed and patted dry with a tissue. After drying with the tissue, step 2 was performed using Composition H of Example 2 (containing lactic acid and salicylic acid). The test composition was allowed to remain on the forearm for 15 minutes, after which the forearm was rinsed and patted dry with a tissue. After drying with the tissue, the forearm was allowed to continue to air dry for 3 minutes.

Tape stripping was carried out on the treated regions of the forearm. A total of 4 tape samples were taken per sampling area (3 sampling areas per treatment regions). After applying tape to the designated area, a metal plate weighting 2 kg was placed on top of the tape for 5 seconds. Then the tape was peeled from the skin. This was repeated 4 times for each sampling area. Each piece of tape (1.4 cm in length) was individually inserted into a 2 mL Eppendorf tube and 500 μl of phosphate buffered saline (PBS) was added. The Eppendorf tubes were shaken at 13,000 rpm for 2 hours at room temperature and then submitted to sonication for twenty minutes in ice water.

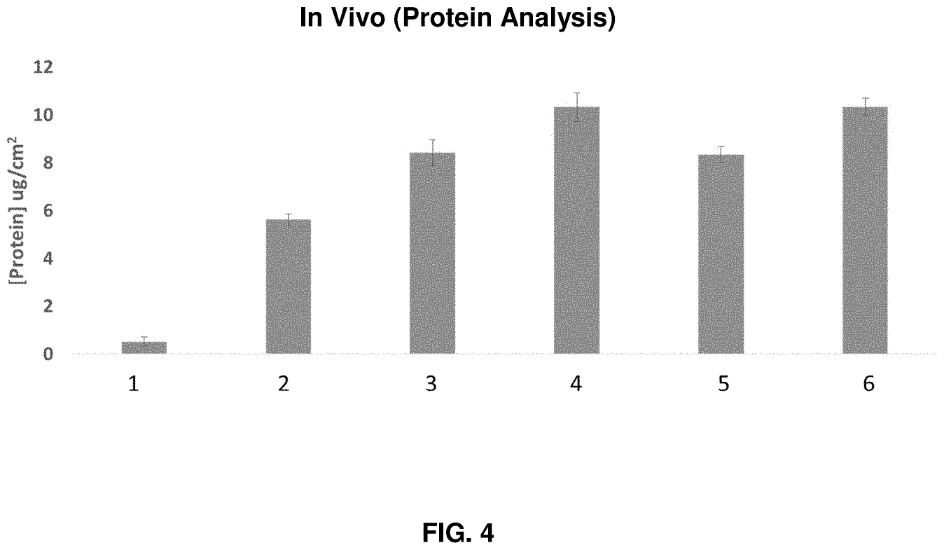

Total protein content recovered by the tape was determined using a micro bicinchoninic acid (BCA) assay. The micro BCA assay quantifies the total amount of protein collected by the tape and therefore represents desquamation efficacy. A higher protein content is associated with a higher desquamation efficacy. The results are reported in the table below and graphically presented in FIG. 4.

| | | | protein | |
|---|---|---|---|---|
| | | | content | |
| Protocol | | Compositions | (μg/ml) | Std |
| 1 | Placebo Control | DI water | 0.5 | 0.26 |
| 2 | Acidic Treatment | Composition H of Example 2 | 5.6 | 0.2 |
| | Only | | | |
| 3 | Alkaline Treatment | Composition B of Example 1 | 8.4 | 0.5 |
| | Followed by Acidic | Followed by Composition H | | |
| | Treatment | of Example 2 | | |
| 4 | Alkaline Treatment | Composition C of Example 1 | 10.3 | 0.6 |
| | Followed by Acidic | Followed by Composition H | | |
| | Treatment | of Example 1 | | |
| 5 | Alkaline Treatment | Composition D of Example 1 | 8.3 | 0.3 |
| | Followed by Acidic | Followed By Composition H | | |
| | Treatment | of Example 1 | | |
| 6 | Alkaline Treatment | Composition A of Example 1 | 10.3 | 0.4 |
| | Followed by Acidic | Followed by Composition H | | |
| | Treatment | of Example 2 | | |

The results show that regardless of the alkaline agent used, the two-step method of first treating the skin with an alkaline composition followed by treatment with an acidic composition surprisingly improved desquamation efficacy.

The foregoing description illustrates and describes the inventions. Additionally, the disclosure shows and describes only the preferred embodiments but it is to be understood that it is capable to use in various other combinations, modifications, and environments and is capable of changes or modifications within the scope of the inventive concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein.

As used herein, the terms “comprising,” “having,” and “including” are used in their open, non-limiting sense.

All percentages, parts and ratios herein are based upon the total weight of the compositions of the present disclosure, unless otherwise indicated.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, less than about 0.05 wt. %, or none of the specified material. The components described for optional inclusion in the compositions of the disclosure may be free of the component(s) or may be “substantially free” or “essentially free” of the component(s).

The term “treat” (and its grammatical variations) as used herein refers to the application of the cosmetic compositions of the present disclosure onto the surface of the body, and in particular the skin and/or hair of the body.

The term “volatile”, as used herein, means having a flash point of less than about 100° C.

The term “non-volatile”, as used herein, means having a flash point of greater than about 100° C.

The term “substituted,” as used herein, means comprising one or more substituents. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

The term “polymers,” as defined herein, include homopolymers and includes copolymers formed from at least two different types of monomers.

The methods and compositions of the present disclosure can comprise, consist of, or consist essentially of the essential elements and limitations of the disclosure described herein, as well as any additional or optional ingredients, components, or limitations described herein or otherwise useful. Additionally, any component that is positively set forth in the present disclosure can be negatively excluded from the methods and compositions of the present disclosure, and in particular from the claims of the present disclosure. In particular, the methods and compositions of the present disclosure can be free or essentially free of any component that is positively set forth in the present disclosure.

Other than in the operating examples, or where otherwise indicated, all numbers expressing quantities of ingredients and/or reaction conditions can be modified, if desired, with the term “about,” meaning within +/−5% of the indicated number.

The terms “a,” “an,” and “the” are understood to encompass the plural as well as the singular. Thus, the term “a mixture thereof” is synonymous with “mixtures thereof.” Throughout the disclosure, the term “a mixture thereof” may be used following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

Some of the various categories of components identified may overlap. In such cases where overlap may exist and the composition includes both components (or the composition includes more than two components that overlap), an overlapping compound does not represent more than one component. For example, a fatty acid may be characterized as both a nonionic surfactant and a fatty compound. If a particular method or composition includes both a nonionic surfactant and a fatty compound, a single fatty acid will serve as only the nonionic surfactant or as only the fatty compound (the single fatty acid does not serve as both the nonionic surfactant and the fatty compound).

All publications, patents, and patent applications cited in the present disclosure are herein incorporated by reference, and for any and all purposes, as if each individual publication, patent, or patent application were specifically and individually indicated to be incorporated by reference. In the event of an inconsistency between the present disclosure and any publications, patents, or patent applications incorporated herein by reference, the present disclosure controls.

1. A method for treating skin comprising:
(a) applying an alkaline composition having a pH of at least 8 to the skin and allowing it to remain on the skin for a first period of time of at least 30 seconds, the alkaline composition comprising:
(i) about 0.05 to about 25 wt. %, based on the total weight of the alkaline composition, of one or more alkaline agents; and
(vi) optionally, about 0.05 to about 1 wt. %, based on the total weight of the alkaline composition, of one or more colorants;
(ii) optionally, about 0.5 to about 25 wt. %, based on the total weight of the alkaline composition, of one or more water-soluble solvents; and
(iii) water; and
(b) after allowing the alkaline composition to remain on the skin for the first period of time, subsequently applying an acidic composition having a pH of 5 or less to the skin and allowing it to remain on the skin for a second period of time of at least 30 seconds, the acidic composition comprising:
(i) about 1 to about 25 wt. %, based on the total weight of the acidic composition, of one or more α- and/or β-hydroxy acids;
(vii) optionally, about 0.1 to about 25 wt. %, based on the total weight of the acidic composition, of one or more whitening actives, such as arbutin, azelaic acid, linoleic acid, kojic acid, N-acetyl-4,S-cysteaminylphenol, niacinamide, resorcinol;
(viii) optionally, about 0.1 to about 25 wt. %, based on the total weight of the acidic composition, of one or more antioxidants, such as ascorbic acid, Vitamin E;
(ix) optionally, about 0.05 to about 1 wt. %, based on the total weight of the acidic composition, of one or more colorants;
(ii) about 1 to about 25 wt. %, based on the total weight of the acidic composition, of one or more water-soluble solvents; and
(iii) water.

2. The method of claim 1, wherein the alkaline composition has a pH of at least 9 and the acidic composition has a pH of 4 or less.

3. The method of claim 1, wherein the first period of time is at least 1 minute and the second period of time is at 1 minute.

4. The method of claim 1, wherein the alkaline composition is not rinsed from the skin prior to application of the acidic composition.

5. The method of claim 1, wherein the alkaline composition is rinsed from the skin prior to application of the acidic composition.

6. The method of claim 1, wherein the one or more alkaline agents of the alkaline composition are selected from:
alkali metal hydroxides,
alkaline-earth metal hydroxides,
alkali metal silicates,
alkali metal carbonates,
alkaline-earth metal carbonates,
organic carbonates,
basic amino acids,
alkanolamines, and
ammonium hydroxide.

7. The method of claim 1, wherein the one or more water-soluble solvents are selected from glycerin, mono-alcohols, polyols (polyhydric alcohols), glycols, and a mixture thereof.

8. The method of claim 1, wherein the alkaline composition is a non-emulsified liquid.

9. The method of claim 1, wherein the alkaline composition is a gel and further comprises:
about 0.1 to about 10 wt. % of one or more thickening agents.

10. The method of claim 9, wherein the one or more thickening agents are selected from carboxylic acid polymers, crosslinked polyacrylate polymers, polyacrylamide polymers, polysaccharides, gums, and a mixture thereof.

11. The method of claim 1, wherein the alkaline composition is an emulsion and further comprises:
about 30 to about 75 wt. % of one or more non-silicone fatty compounds; and
about 0.1 to about 10 wt. % of one or more emulsifiers.

12. The method of claim 11, wherein the one or more non-silicone fatty compounds are selected from oils, mineral oil, alkanes (paraffins), fatty alcohols, fatty acids, fatty alcohol derivatives, fatty acid derivatives (such as alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof.

13. The method of claim 11, wherein the one or more emulsifiers are selected from alkylpolyglycosides, glyceryl esters, ethylene glycol esters, propylene glycol esters and sucrose esters of fatty acids, ethoxylated fatty alcohols, ethoxylated fatty acids, partial glycerides of ethoxylated fatty acids, polyglycerolated fatty acid triglycerides, and a mixture thereof

14. The method of claim 1, wherein the one or more α- and/or β-hydroxy acids of the acidic composition are selected from lactic acid, glycolic acid, citric acid, ascorbic acid, phytic acid, mandelic acid, and salicylic acid.

15. The method of claim 1, wherein the one or more water-soluble solvents of the acidic composition are selected from glycerin, mono-alcohols, polyols (polyhydric alcohols), glycols, and a mixture thereof.

16. The method of claim 1, wherein the acidic composition is a non-emulsified liquid.

17. The method of claim 1, wherein the acidic composition is a gel and further comprises:
about 0.1 to about 10 wt. % of one or more thickening agents.

18. The method of claim 17, wherein the one or more thickening agents are selected from carboxylic acid polymers, crosslinked polyacrylate polymers, polyacrylamide polymers, polysaccharides, gums, and a mixture thereof.

19. The method of claim 1, wherein the acidic composition is an emulsion and further comprises:
about 5 to about 50 wt. % of one or more non-silicone fatty compounds; and
about 0.1 to about 10 wt. % of one or more emulsifiers.

20. The method of claim 19, wherein the one or more non-silicone fatty compounds are selected from oils, mineral oil, alkanes (paraffins), fatty alcohols, fatty acids, fatty alcohol derivatives, fatty acid derivatives (such as alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof.

21. The method of claim 20, wherein the one or more emulsifiers are selected from alkylpolyglycosides, glyceryl esters, ethylene glycol esters, propylene glycol esters and sucrose esters of fatty acids, ethoxylated fatty alcohols, ethoxylated fatty acids, partial glycerides of ethoxylated fatty acids, polyglycerolated fatty acid triglycerides, and a mixture thereof

22. The method of claim 1, wherein the acidic composition comprises at least 10 to about 25 wt. %, based on the total weight of the acidic composition, of the one or more α- and/or β-hydroxy acids.

23. The method of claim 1, wherein the method provides desquamation of the skin of the face.

24. The method of claim 1, wherein the method:
improves the radiance of skin;
improves the evenness of skin tone;
improves the clarity of skin; and/or
improves the overall appearance of skin.
